(19) United States
(12) Patent Application Publication
(10) Pub. No.: US 2026/0315729 A1
(43) Pub. Date: Oct. 15, 2026

(54) WEARABLE DEVICE FOR MITIGATING SYMPTOMS OF NEUROLOGICAL CONDITIONS OF USERS AND METHOD FOR USING SAME

(71) Applicant: CHARCO NEUROTECH LTD, Cambridge (GB)

(72) Inventors: Soo Min Jung, Cambridge (GB); Floyd Pierres, Cambridge (GB); Ki Cheol Park, Cambridge (GB); Archit Parhi, Cambridge (GB); Hassaan-Ul Huda, Cambridge (GB)

(21) Appl. No.: 19/485,328

(22) PCT Filed: May 20, 2024
(86) PCT No.: PCT/IB2024/054886

§ 371 Date: Nov. 18, 2025

(30) Foreign Application Priority Data
May 19, 2023 (GB) 2307532.8
May 19, 2023 (GB) 2307557.5

(51) Int. Cl. A61M 21/00

(52) U.S. Cl. CPC A61M 21/00; A61M 2021/0022; A61M 2021/0027; A61M 2205/3303; A61M 2209/088

(57) ABSTRACT

There is provided a wearable device (100, 200, 300, 400, 600, 700, 802) for mitigating symptoms of a neurological condition of a user (202, 302, 602), wherein the wearable device includes: a mounting arrangement (102) for detachably mounting the wearable device to the user; a power source (104) for providing electrical power to the wearable device when in use. The wearable device (100, 200, 300, 400, 600, 700, 802) comprises moreover: a stimulation arrangement (106) for applying a stimulation to the user, wherein the stimulation includes vibrational stimulation or auditory stimulation, or both vibrational stimulation and auditory stimulation, a sensor arrangement (110) for sensing characteristics of the user to generate an input signal; wherein the sensor arrangement includes one or more motion sensors and/or one or more biosensors to sense a motion patterns and/or a biological patterns of the user (202, 302, 602), wherein the motion patterns and/or biological patterns are associated with the symptoms of the neurological condition of the user (202, 302, 602); and a control arrangement (108) for processing the input signal to generate a corresponding output signal for driving the stimulation arrangement (106) for applying the stimulation to the user. The wearable device is configured to excite the stimulation arrangement to provide pulsing period stimulation (VS2, AS2) during a pulsing period and to provide resting period stimulation (VS1, AS1) during a resting period, wherein the pulsing period stimulation (VS2, AS2) is greater in amplitude than the resting period stimulation (VS1, AS1), wherein the pulsing period and the resting period are included within a cueing period, wherein the wearable device is configured to provide a concatenated series of such cueing periods, and wherein the wearable device is configured to provide cueing.

### TECHNICAL FIELD

The present disclosure relates to wearable devices for mitigating symptoms of neurological conditions of users. Moreover, the present disclosure relates to methods for (namely, methods of) using aforementioned wearable devices for mitigating symptoms of neurological conditions of users. Furthermore, the present disclosure relates to software products that are executable on computing arrangements including computing hardware for implementing aforementioned methods.

### BACKGROUND

People who suffer from neurological conditions, for example, stroke, Parkinson's disease or injury, often experience difficulties in movement. Such difficulties include (but are not limited to) gait, hesitation, stiffness, freezing-of-gait (FoG), slowness, stutter or stammer, forward flexion of trunk (FFT), and tremors or shaking. Whereas gait is a type of walking and associated movement, different from normal healthy walking and associated movements, that people exhibit, tremors are involuntary shaking that may occur in the limbs of people when they are suffering from neurological conditions. Often such difficulties in movement impact the ability of such people to carry out normal daily activities.

Conventional approaches for dealing with the symptoms of such conditions typically utilize medication or deep brain stimulation, both of which are complex, costly and require a trained medical professional to supervise associated mitigating treatments. Therefore, non-pharmacological and non-invasive approaches to mitigate symptoms of such conditions are conventionally preferred. In this regard, over the recent past, environmental stimulation, for example, vibrotactile (namely, skin-contact vibrational) stimulation of the skin, and corresponding stimulation of the human brain resulting therefrom, has been gaining global attention. It is hypothesized that the human brain exhibits neuroplasticity; in other words, vibrotactile stimulation to skin of a user causes the user's human brain to reconfigure itself, resulting in suppression of symptoms of the user's neurological conditions, for example the user's neurological conditions that give rise to gait. By using such an approach of using vibrotactile stimulation to induce changes in brain function through neuroplasticity, it is feasible to enable a given person to walk more normally without tremor, hesitation or freezing-of-gait (FOG).

Moreover, conventional vibrotactile stimulation devices need to be arranged to contact the body of the user to impart vibrotactile stimulation thereto. In this regard, the vibrotactile stimulation device may be contacted with the body of the user by using adhesives or by using an adhesive patch therebetween. However, this is often not a reliable solution for people who are more active in pursuits such as sports, and so on. Moreover, the adhesive may lose adhesion with time, requiring regular reapplication of the adhesive and thus increasing an overall monthly expense to the user using the conventional vibrotactile stimulation devices.

Moreover, currently, various non-invasive techniques are used to identify biological patterns and characteristics of a given user (for example, a person). For example, Magnetic Resonance Imaging (MRI) is a non-invasive imaging technique that uses strong magnetic fields and radio waves to generate detailed images of a brain and a spinal cord of a given user. However, such an MRI is potentially expensive and may not be suitable for users with certain medical conditions, such as severe claustrophobia. Similarly, an electroencephalogram (EEG) is a non-invasive test that measures an electrical activity of a brain of a given user through use of electrodes that are placed on a scalp of the given user, wherein the electrodes enable measurements to be made in real-time. However, such an EEG may not be suitable for users with hair or scalp conditions that interfere with electrode placement.

Therefore, in light of the foregoing discussion, there exists a need to overcome the aforementioned drawbacks.

### SUMMARY

The aim of the present disclosure is to provide a wearable device, and a method for (namely, a method of) using the wearable device to provide stimulation with cueing to mitigate physical symptoms associated with a medical condition, such as a neurological condition, of a user; “cueing” is a particular form of treatment that is at least partially synchronized to movements of a given user who is using the wearable device. The aim of the present disclosure is achieved by using a wearable device and a method for operating the aforementioned wearable device for mitigating symptoms of a neurological condition of a user as defined in the appended independent claims to which reference is herewith made. Advantageous features are set out in the appended dependent claims.

In a first aspect, the present disclosure provides a wearable device for mitigating symptoms of a neurological condition of a user, according to claim 1.

In a second aspect, the present disclosure provides a method for (namely, a method of) operating a wearable device for mitigating symptoms of a neurological condition of a user, according to claim 23.

In a third aspect, an embodiment of the present disclosure provides a software product that is executable on a computing arrangement including computing hardware for implementing the method of the second aspect, wherein the software product is according to claim 24.

Throughout the description and claims of this specification, the words “comprise”, “include”, “have”, and “contain” and variations of these words, for example “comprising” and “comprises”, mean “including but not limited to”, and do not exclude other components, items, integers or steps not explicitly disclosed also to be present. Moreover, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

FIGS. 1A and 1B are illustrations of temporal details of various stimulations that are applied to a given user by a wearable device of the present disclosure, when in use, for example during “cueing”;

FIG. 2 is an illustration of a form of output signal that excites vibrational stimulation in a wearable device of the present disclosure;

FIG. 3 is an illustration of a block diagram of a wearable device for mitigating symptoms of a neurological condition of a user, according to an embodiment of the present disclosure;

FIGS. 4A and 4B are illustrations of a front view of a wearable device arranged on a strap arrangement for mitigating symptoms of a neurological condition of a user, according to an embodiment of the present disclosure;

FIGS. 5A and 5B are illustrations of a front view and a side view of a wearable device arranged on a strap arrangement for mitigating symptoms of a neurological condition of a user, according to an embodiment of the present disclosure;

FIGS. 6A and 6B are illustrations of exemplary implementations of a wearable device arranged on a strap arrangement for mitigating symptoms of a neurological condition of a user, according to an embodiment of the present disclosure;

FIG. 7 is an illustration of an exploded view of a linear resonant actuator (LRA), according to an embodiment of the present disclosure;

FIG. 8 is an illustration of a front view of a wearable device for mitigating symptoms of a neurological condition of a user, according to an embodiment of the present disclosure;

FIG. 9 is an illustration of a wearable device for mitigating symptoms of a neurological condition of a user, according to an embodiment of the present disclosure;

FIG. 10 is an illustration of an exploded view of an overhead adhesive patch, according to an embodiment of the present disclosure;

FIG. 11 is a flowchart depicting steps of a method for operating a wearable device for mitigating symptoms of a neurological condition of a user, according to an embodiment of the present disclosure;

FIG. 12 is a schematic block diagram of a database system, implemented as a software-as-a-service (Saas) or platform-as-a-service (PaaS), for supporting one or more wearable devices according to the present disclosure; and

FIG. 13 is an illustration of characteristics of operation of a linear resonant actuator (LRA), as used in embodiments of the present disclosure.

### DETAILED DESCRIPTION OF EMBODIMENTS

The following detailed description illustrates embodiments of the present disclosure and ways in which they may be implemented. Although some modes of carrying out the present disclosure have been disclosed, those skilled in the art would recognize that other embodiments for carrying out or practising the present disclosure are also possible.

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In a first aspect, the present disclosure provides a wearable device for mitigating symptoms of a neurological condition of a user, wherein the wearable device includes:
a mounting arrangement for detachably mounting the wearable device to the user;
a power source for providing electrical power to the wearable device when in use;
a stimulation arrangement for applying a stimulation to the user, wherein the stimulation includes vibrotactile stimulation or auditory stimulation, or both vibrotactile stimulation and auditory stimulation;
a sensor arrangement for sensing characteristics of the user to generate an input signal; wherein the sensor arrangement includes one or more motion sensors and/or one or more sensors to sense a motion patterns and/or a biological patterns of the user, wherein the motion patterns and/or biological patterns are associated with the symptoms of the neurological condition of the user;
control arrangement for processing the input signal to generate a corresponding output signal for driving the stimulation arrangement for applying the stimulation to the user,
wherein the wearable device is configured to excite the stimulation arrangement to provide pulsing-period stimulation (VS2, AS2) during a pulsing period and to provide resting-period stimulation (VS1, AS1) during a resting period, wherein the pulsing-period stimulation (VS2, AS2) is greater in amplitude than the resting-period stimulation (VS1, AS1), wherein the pulsing period and the resting period are included within a cueing period,
wherein the wearable device is configured to provide a concatenated series of such cueing periods, and
wherein the wearable device is configured to provide cueing.

Beneficially, the stimulation is limited to a specific part of the user's body; in other words, the stimulation provided by the wearable device is “focused”.

Beneficially, the wearable device provides vibrotactile stimulation or auditory stimulation, or both, (namely, vibrotactile simulation and/or auditory stimulation) with cueing to the user with physical symptoms associated with neurological disorders or any other medical condition. The wearable device of the present disclosure makes use of a combination of pulsing stimulation and resting stimulation to generate the stimulation with cueing to help the user to mitigate his/her neurological symptoms. For acceptance by the user, it is beneficial that the wearable device is aesthetically appealing, besides being extremely comfortable for the user to wear. Moreover, the stimulation is beneficially implemented to be comfortable to the user, in terms of noise control, heat dissipation, etc. The wearable device is tailored to suit the requirements of the user, such as in terms of adjusting parameters of the stimulation to refine the pattern, amplitude and frequency of stimulation from which the user experiences greatest benefits. The wearable device is capable, for example, of mitigating symptoms of impaired gait (i.e. a manner of walking and standing) and thereby providing a better quality of life to the user. The wearable device provides a non-invasive treatment, and the impact of vibrotactile stimulation and/or auditory stimulation produced thereby may be extended in the context of rehabilitation programmes and demonstrate long term benefits. The wearable device is able to induce changes in the user's brain related to neuroplasticity and increase responsiveness to the vibrotactile and/or auditory stimulations. Furthermore, the wearable device is a stand-alone device and may be operated by device integration application software for recording and analysing the overall health (namely, quality of life (QoL)) of the user.

Throughout the present disclosure, the term “wearable device” as used herein refers to a device that is configured to mitigate, alleviate or eliminate physical symptoms associated with one or more medical conditions. Optionally, the medical condition may be a neurological condition (such as Parkinson's Disease (PD), stroke, spinal cord injury, and so on), multiple sclerosis, fibromyalgia, and so on. Typical symptoms associated with such medical conditions include, but are not limited to, stiffness, hesitation, slowness, freeze-of-gait (FOG), freeze-of-movement, balance problems, numbness or tingling in limbs, tremors or shaking, fatigue, and stuttering, sleep disturbances. In an exemplary implementation, the wearable device may optionally be used to mitigate the symptoms such as tremors, freeze-of-gait (FoG) and hesitation associated with Parkinson's disease. In another exemplary implementation, the wearable device may be optionally used to mitigate the symptoms such as numbness or tingling in limbs associated with multiple sclerosis.

Therefore, in order to mitigate such symptoms, the wearable device is beneficially arranged to make physical contact with the user's body, namely to a region of skin on the user, by a mechanical engagement, and wherein the mechanical engagement may be by way of at least one of: a strap, an adhesive, a locket, a bracelet, a band, a belt, a vacuum cup, a magnet. The wearable device may be placed against any body part of the user such as the user's sternum, arm, shoulder, wrist, neck, ankle, leg, hip and temple or head region. It will be appreciated that the aforementioned body parts allow for easy access of the wearable device by the user and by a carer or family member of an incapacitated user. Some users may not be comfortable in showing off the wearable device and may correspondingly wear the wearable device at any body part hidden under a piece of cloth, or the strap arrangement, for example.

The wearable device includes a mounting arrangement for detachably mounting the wearable device to the user. The term “mounting arrangement” as used herein refers to a means for attaching or securing the wearable device to contact the body of the user, for example via use of a strap arrangement, adhesive patches and so forth. In this regard, it will be appreciated that the strap arrangement may be implemented as a receptor (or receptacle) for the wearable device. Herein, the mounting arrangement may include any type of attachment mechanism, such as straps, Velcro® fasteners, clips, bolts, screws, brackets, clamps, or magnets that are configured to attach and detach the wearable device to and from the strap arrangement. Optionally, the mounting arrangement of the wearable device is complementary with a mounting arrangement of the strap arrangement. Herein, the term “complementary” refers to suitably sized and shaped counterparts that when come together as a unit form a secured arrangement. In an example, the mounting arrangement of the wearable device and the strap arrangement are implemented using a Velcro® fastener, as aforementioned. Velcro® fasteners are a type of hook-and-loop fastener that comprises a hook layer and a loop layer that are configured to couple to each other by means of tiny hooks and smaller loops therein, respectively; when pressed together, the hooks catch in the loops and the two layers fasten or bind temporarily. The two layers are separated by pulling or peeling apart the loop layer from the hook layer. Beneficially, a suitable mounting arrangement is important for ensuring that the wearable device stays securely in place and may be comfortably worn for extensive periods of time by the user.

The wearable device includes a power source for providing electrical power to the wearable device, when in use. The power source is optionally implemented as a rechargeable and/or replaceable battery that is configured to power the wearable device, when in operation. Optionally, the wearable device is charged at a charge rate in a range of 53 milliampere (mA) to 110 milliampere (mA) when a voltage of 5V is applied thereto. Optionally, the voltage is in a range of 2.5V to 5V, more optionally, 2.9V to 3V, yet more optionally substantially 3V. Moreover, at such voltage conditions, the LEDs are driven to a maximum current of 5 mA.

Furthermore, the wearable device includes the stimulation arrangement for applying vibrotactile stimulation and/or auditory stimulation (note: “and/or” means “at least one of”, for example “both”) to the user, wherein the stimulation is optionally implemented in a pulsed manner. It will be appreciated that the wearable device is optionally configurable to provide continuous stimulation, if required. Moreover, it will be appreciated that the stimulation may also include, or be, a stimulation pressure or a change in stimulation pressure, a rolling motion, a tap or other impact, etc. Notably, the stimulation is optionally received as a force that affects muscles and neurons of the user. Herein, the stimulation may, for example, refer to a plurality of modes of stimulations suitable for a given user and to mitigate a specific level of symptom associated with his/her medical condition.

The wearable device may also comprise a dissipating portion configured to increase an effective area of vibrotactile stimulation provided by the stimulation arrangement to be delivered to the user. Optionally, the dissipating portion comprises a flexible or elastic material, for example, a viscoelastic material, or a viscoelastic polymeric material such as a silicone, rubber, flexible plastics material, foam, and the like. The dissipating portion forms at least a part of a proximal surface of the wearable device configured to contact, either directly or indirectly, the body of the user. Herein, the proximal surface is configured to deliver and/or transmit the vibrotactile stimulation from the stimulation arrangement to the user.

Furthermore, the wearable device includes the sensor arrangement for sensing characteristics of the user to generate the input signal (Vᵢₙ(t)). Throughout the present disclosure, the term “sensor arrangement” refers to an arrangement of one or more sensors that are configured to sense data that relates to specific characteristics associated with the body of the user. Notably, the characteristics that are sensed is indicative of a severity of the symptoms of the neurological condition. Herein, the input signal (Vᵢₙ(t)) is an electrical signal generated based on the sensed data of the characteristics of the user and includes information about the severity of the symptoms of the neurological condition.

Furthermore, the wearable device includes the control arrangement for generating the output signal for driving the stimulation arrangement for applying stimulation to the user. The term “control arrangement” as used herein refers to a hardware, software, firmware or a combination of these, suitable for controlling the operation of the wearable device. The control arrangement is communicably coupled to the stimulation arrangement and the power source and optionally to an external device associated with the wearable device. Examples of the control arrangement include, but are not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the control arrangement may refer to one or more individual controllers, serving devices and various elements associated with a serving device that may be shared by other serving devices.

Herein, the wearable device that incorporates a vibrotactile stimulation motor may generate a vibrotactile stimulation by receiving an output signal from the control arrangement that excites the vibrotactile stimulation motor. The control arrangement may be programmed to generate different output signals that control the frequency, amplitude, and duration of the vibrotactile stimulation, tailored to characteristics of the user and the magnitude of his/her symptoms.

The term “output signal” as used herein refers to an electrical or electronic signal generated by the control arrangement and is configured, for example, to drive or control the stimulation arrangement to enable a vibration motor or a set of vibrotactile motors to produce vibrotactile stimulation to be applied to the user; optionally, only a single vibration motor is used. Optionally, the output signal may include voltage signals, current signals, digital signals, or analog signals.

Optionally, the components of the wearable device are suitable for a desired temperature and humidity, such as suitable for moisture sensitivity level (MSL) of 1 and the operating temperature in a range of 0 °C to 125 °C, optionally, −40 °C to 85 °C, and more optionally, −40 °C to 125 °C.

Optionally, the aforesaid wearable device is included as a part of a kit of parts when provided to the user. The kit of parts may comprise the wearable device and a docking station. The docking station may comprise a recess configured to receive the wearable device; for example, the user may park the wearable device in the docking station for storage or recharging when the wearable device is not being worn by the user. Moreover, the recess comprises a receiving surface for receiving the wearable device, and also comprises a surrounding wall that is configured to prevent movement of the wearable device on the receiving surface. The docking station may be configured to transmit electrical power wirelessly via a wireless charging transmitter, for example implemented as one or more coils or windings of electrically conductive material, to charge the rechargeable power source, when the wearable device is received within the recess of the docking station. Additionally, the docking station may be configured to connect to an external power source (for example, mains electricity) using a wired connection to provide electric power (for example, to the wireless charging transmitter) to be wirelessly transmitted to the wearable device.

Optionally, the docking station comprises a single body having a lid attached at one end to a base thereof. The base and the lid may be connected together to form a casing for the wearable device. The casing may protect the wearable device during storage or transit. The base and the lid may be releasably attachable, for example using corresponding male and female engagement features such as a press-fit or interference fit, elastic clips and flanges, or complementary screw threads; such an attachment is optionally implemented as a simple flip motion, because some users may have physical difficult to twist objects. The base and the lid may form an internal recess of the docking station to securely enclose the wearable device within the casing when the base and the lid are connected or attached together. The base may comprise the recess configured to receive the wearable device and the wireless charging transmitter. The recess may be included in an outer upper surface of the base. The base may rest on or stand on the lower surface in normal use. Optionally, the base may comprise a reservoir for containing additional overhead adhesive patches for later use by the user; alternatively, such additional overhead adhesive patches may be provided in a separate container relative to the base, wherein the base merely includes original bottom adhesive patches. Optionally, the lid may not be connected to the base in normal use; for example, the lid may be detachable when the wearable unit is received within the recess to receive electric power wirelessly from the docking station. Optionally, the lid may be connected to the base to store or transport the wearable device. Alternatively, the reservoir may be included in the lid of the docking station.

An outer upper surface (for example, an end wall or a side wall) of the docking station may be removable from the docking station to provide access to the reservoir (for example, for the user). The reservoir may be refillable with the additional overhead adhesive patches. A size of the outer surface that is removable (by rotating outwards the part of the outer upper surface containing the reservoir) from the docking station may be large enough to allow the wearable device (for example, a distal surface configured to contact with the strap arrangement, and arranged opposite to the proximal surface configured to deliver or transmit the vibrational stimulation to the user) to pass into the reservoir and contact the overhead adhesive patches in the reservoir. This may enable easy application of the additional overhead adhesive patches to the wearable device as and when required.

Moreover, optionally, the one or more overhead adhesive patches are provided to the reservoir in distinct, separate layers (for example, a stack of overhead adhesive patches) which are individually retrievable from the reservoir and which may be applied individually (or separately) to the wearable device. The one or more overhead adhesive patches are arranged in the reservoir such that the side corresponding (or attachable) to the surface of the wearable device is exposed from the reservoir. When the wearable device is pressed in the reservoir, overhead adhesive patches (a top layer, referred to as a hook layer hereafter) are transferred onto the surface of the wearable device. The other (or bottom layer, referred to as a loop layer hereafter) overhead adhesive patches are retained within the reservoir during this process. The overhead adhesive patches comprise a peelable cover with a tab to enable easy removal of the cover. Optionally, the adhesive patches include electrodes for use as biosensors for sensing biological characteristics of the user.

Each of the overhead adhesive patches may be implemented as a hook-and-loop fastener, such as a Velcro® fastener, wherein the patches include adhesive. Operation of Velcro® fasteners is described in the foregoing. The hook-and-loop fasteners comprise a hook layer and a loop layer. The hook layer and the loop layer are configured to couple to each other by means of tiny hooks in the hook layer and smaller loops in the loop layer. The respective backs of the hook layer and the loop layer comprise adhesives. Adhesives on the hook layer and the loop layer may be of different grades and strength. The overhead adhesive patches comprise a first layer of adhesive coupled to the hook layer and a second layer of adhesive coupled to the loop layer. The overhead adhesive patch is arranged to attach to the strap arrangement by the second layer of adhesive coupled to the loop layer and to the surface of the wearable device by the first layer of adhesive coupled to the hook layer.

The wearable device may be placed onto the body of the user by using the overhead adhesive patches, wherein the distal surface of the wearable device is coupled to the strap arrangement, so that the proximal surface of the wearable device contacts the body of the user. The wearable device may be removed for charging or when not required for use, with the loop layer of the overhead adhesive patch remaining attached on the strap arrangement via the second layer of adhesive, while the hook layer of the overhead adhesive patches remains attached on the wearable device via the first layer of adhesive. This allows using the same overhead adhesive patch for a predefined time, while the wearable device is being charged. Eventually, when the overhead adhesive patch has been used for the required number of days, for example 14 days or more, such as 20 days or more, the overhead adhesive patch may be removed and a new overhead adhesive patch may then be applied using the process described above. Alternatively, the overhead adhesive patch may be replaced if the overhead adhesive patch fails to provide desired adhesion between the strap arrangement and the wearable device. Moreover, the hook layer is beneficially strong enough to last several months, if not years. However, the hook layer may also be optionally detached from the wearable device in case the hook layer wears off.

Beneficially, the wearable device provides vibrotactile stimulation to the users with physical symptoms associated with neurological disorders or another medical condition; optionally, the wearable device provides other types of stimulation in addition to, or as an alternative to the vibrotactile stimulation, for example auditory stimulation, electrical stimulation, thermal stimulation, light stimulation, but not limited thereto. The wearable device of the present disclosure beneficially uses a combination of vibrotactile stimulation and/or auditory stimulation with cueing during a pulsing period and a resting period to generate the stimulation in a focused manner (referred to as “focused high frequency vibrotactile and/or auditory stimulation and cueing, wherein the stimulation is applied to a specific portion of the user”) to help the user to mitigate his/her symptoms. The wearable device is tailored to suit the requirements of the user, such as in terms of altering variables of the stimulation to refine a pattern, amplitude and frequency of the stimulation from which the user experiences greatest benefits.

Herein, the “pulsing periods” refer to periods of time during which the wearable device provides pulsing vibrotactile and/or auditory stimulation to the user. The purpose of the pulsed stimulation is to prompt the user to take a certain action or engage in a particular behavior. The exact nature of the pulsing-period stimulation beneficially depends on the specific design of the wearable device, but it may optionally be some kind of physical or sensory feedback provided to the user, such as vibrational and/or auditory cues. During the pulsing periods, the wearable device is actively strongly exciting the stimulation arrangement; in contradistinction, during the resting period, the wearable device is actively weakly exciting the stimulation arrangement (or potentially even ceasing to excite the stimulation arrangement if VS1=0, as aforementioned). Herein, the resting period refers to a period of time during which the wearable device substantially ceases to provide stimulation to the user, or provides only weak stimulation to the user.

Optionally, the control arrangement is configured to excite the stimulation arrangement to provide vibrotactile stimulation within a frequency range of 20 Hz to 500 Hz during the pulsing period, optionally in a range of 30 Hz to 300 Hz, optionally in a range of 80 Hz to 200 Hz, although other frequency ranges are optionally used. The technical effect of limiting the stimulation arrangement to be excited within a range of specific frequencies within the 20 Hz to 500 Hz range is that it is the most effective type of vibrotactile stimulation to provide for the user. Such a range of frequencies results in improved efficacy and customization of the wearable device for mitigating symptoms of the user's neurological condition.

Optionally, the wearable device is configured to implement the cueing period to have a duration in a range of 0.2 seconds to 5 seconds, more optionally in a range of 0.2 seconds to 4 seconds. The cueing period is chosen in cueing to coincide with a manner of movement of the user, for example as aforementioned.

Optionally, the stimulation arrangement is configured to apply the at vibrotactile stimulation during the pulsing period to have a frequency that is independently adjustable to an amplitude of the vibrotactile stimulation. In other words, the frequency of the vibrotactile stimulation may be independently adjusted to the amplitude of the vibrotactile stimulation during the pulsing period. The technical effect of this feature is that it allows for greater customization and optimization of the vibrotactile stimulation delivered to the user. By adjusting the frequency of the stimulation relative to the amplitude of the stimulation, the user may receive a more precise and effective treatment. This may be particularly useful in therapeutic applications where specific frequencies and amplitudes are known to have beneficial effects on the user's body.

Optionally, the stimulation arrangement includes at least one actuator, for example just a single actuator, that may be vibrationally coupled to a skin region of the user. Additionally, coupling the at least one actuator directly to the skin region may improve the efficiency of delivery of the vibrotactile stimulation by reducing the amount of energy that is lost as the vibration travels through layers of clothing or other materials.

Optionally, the at least one actuator includes at least one of: a linear resonant actuator (LRA), an eccentric rotating mass (ERM) DC motor, an eccentric rotating mass (ERM) a brushless direct current (DC) motor (BLDC), a piezo-electric actuator; optionally, there is used only a single actuator on the wearable device 100. The term “linear resonant actuator” as used herein refers to a vibration motor that produces an oscillating force, optionally along a single axis. The linear resonant actuator optionally comprises a resonant spring-mass system that is mechanically excited by using a solenoid coil, for example implemented as a voice coil of a loudspeaker. In use, the linear resonant actuator relies on an alternating current (AC) voltage to drive the solenoid coil (for example, voice coil) pressed against a movable mass that is supported by a spring. In this regard, when the solenoid coil (for example, voice coil) is driven near or below the resonant frequency (“natural frequency”) of the spring-mass system comprising the spring and the movable mass, the entire actuator vibrates with a perceptible force; optionally, the perceptible force is in a range of 0.1 G to 2.0 G. By driving the movable mass up and down against the spring, the linear resonant actuator as a whole will be displaced and produce the vibrotactile stimulation. Beneficially, the linear resonant actuator reduces power consumption by taking advantage of the resonant frequency of the spring-mass system. Since the solenoid coil (for example, voice coil) is driven by the alternating current (AC), the desired frequency and amplitude of vibration may be independently varied in use. Beneficially, the technical effect of including the linear resonant actuator in the stimulation arrangement is that the linear resonant actuator does not include fast-rotating parts that are prone to wear; for example, brushes within a rotating DC motor (ERM DC motor) are particularly prone to wear. Thus, the linear resonant actuator is capable of providing a longer service life to the wearable device, when in use.

Optionally, the wearable device includes only a single actuator that is configured to provide the vibrotactile stimulation over a frequency range of interest, for example from 80 Hz to 200 Hz. Alternatively, optionally, the at least one actuator includes a plurality of linear resonant actuators whose resonant frequencies are mutually different, to provide the stimulation arrangement in aggregate with an extended frequency operating range with high operating efficiency. Optionally, the at least one actuator includes two or more linear resonant actuators that are oriented in different directions of motion in the wearable device. It will be appreciated that the resonant frequencies of the plurality of linear resonant actuators are tuned in a manner that they are mutually different, which provides the wearable device with the broader aggregate stimulation frequency range when providing the vibrotactile stimulation. For example, the wearable device includes two linear resonant actuators, one with a resonant frequency of 100 Hz and the other with a resonant frequency of 200 Hz. In such a case, when the two linear resonant actuators are used together, they can provide the broader aggregate stimulation frequency response range from 100 Hz to 200 Hz.

Optionally, the at least one linear resonant actuator has a Q-factor of resonance in a range of 2 to 10, more optionally in a range of 2 to 5. The technical effect of having the one or more linear resonant actuators with the Q-factor in this range is that it allows for the creation of the wearable device that is able to provide the vibrotactile stimulation over a wider range of frequencies while maintaining efficiency of operation (for example, over a frequency range including a resonant frequency and at frequencies below the resonant frequency).

Optionally, the wearable device is configured to include a conformal cushioning element between the wearable device and a strap arrangement used to support the wearable device onto the user's skin, wherein the conformal cushioning element is beneficially omitted from a region wherein the at least one actuator provides in use its vibrotactile stimulation to the skin region of the user. The term “conformal cushioning element” as used herein refers to a substance or material that interacts with the user's body in a way that preserves the underlying conformal symmetry (such as angles and shapes of user's body under a conformal transformation) thereof.

Beneficially, the cushioning element has a purpose to fill a gap between the strap arrangement and the wearable device, because the human chest or sternum area may have very different shapes, such as concave or convex chest, from one user to another.

Optionally, the conformal cushioning element is implemented as a memory foam. The term “memory foam” as used herein refers to a type of foam that has the ability to contour to the shape of an object when it is under pressure and then return to its original shape when the pressure is removed. Optionally, the memory foam includes additional chemicals that increase viscosity and density thereof. Typically, the memory foam possesses viscoelastic properties, that allows the memory foam to respond to heat and pressure. Generally, bubbles or ‘cells’ of the memory foam are open, effectively creating a matrix through which air is able to move. Moreover, when the memory foam has a higher density, it softens in reaction to the user's body heat, thereby allowing the memory foam to mould to a warm body in a few minutes (such as in a range of 1 minute to 3 minutes).

Optionally, the memory foam derives its viscoelastic properties from several effects that occur due to the internal structure thereof. For example, a network effect is a force working to restore the memory foam's structure when it is deformed. In this regard, such a network effect is generated by the deformed porous material pushing outwards to restore its structure against an applied pressure. Typically, there exist three effects such as a pneumatic effect, an adhesive effect and a relaxation effect that work against the network effect, slowing the regeneration of the memory foam's original structure. Moreover, the pneumatic effect is caused by the time it takes air to flow into the memory foam's porous structure. The adhesive effect, or adhesion, is caused by the stickiness of the surfaces within the memory foam that works against decompression as the internal pores within the memory foam are pressed together. The relaxation effect (the strongest of the aforementioned three forces working against expansion) is caused by the memory foam's material being near to its glass transition temperature, thus limiting mobility thereof, forcing any change to be gradual, and slowing the expansion of the memory foam once the applied pressure has been removed. The technical effect of using the memory foam is that it provides a custom-fit and comfortable abutting surface for the user.

Optionally, the conformal cushioning element is manufactured from a polymer foam, optionally polyurethane foam. Herein, the term “polyurethane foam” refers to a type of polymer foam that is widely used in many applications due to its high resilience, durability, and ability to conform to different shapes and sizes.

Optionally, the wearable device further includes the user interface arrangement for the user to provide a trigger signal, for example by pressing a button, for the wearable device to function to provide the stimulation with cueing; “cueing” is described in greater detail in the foregoing. Throughout the present disclosure, the term “user interface arrangement” refers to a means that enables the user to control and communicate with the wearable device. The user interface arrangement provides a way for the user to provide the trigger signal.

Optionally, the user interface arrangement may be implemented as a button installed in the wearable device. Alternatively, the user interface is implemented as a touchpad of a display screen installed in the wearable device.

Throughout the present disclosure, the term “trigger signal” refers to a signal that is used to trigger the wearable device to function when required by the user. Notably, the user is able to provide the trigger signal via the user interface arrangement (i.e., via pressing the button installed in the wearable device, or via clicking on the touchpad of the display screen installed in the wearable device). It will be appreciated that the trigger signal may be provided by the user at a time instance when the user feels the symptoms of his/her neurological condition building up in his/her body. Subsequently, once the trigger signal is provided, the wearable device functions to provide the stimulation, namely vibrotactile stimulation and/or auditory stimulation with cueing, for example; “and/or” is to be construed as defined in the foregoing. The technical effect of using the user interface arrangement to provide the trigger signal enables the user to control when to use the wearable device according to the user's requirements.

Optionally, the wearable device is configured such that the sensor arrangement includes one or more motion sensors to sense motion patterns of the user. The one or more motion sensors enable to detect motion parameters or patterns of the user, wherein the motion patterns are associated with the symptoms of the neurological condition of the user. In this regard, a motion sensor is a device that senses one or more motion parameters or patterns, such as user's speed, the user's acceleration, the user's orientation, and the like, by converting the motion pattern into a corresponding electrical signal. Moreover, the one or more motion sensors may be used to select the cueing period and thus the cueing frequency for the user based on the motion patterns of the user sensed by the one or more motion sensors.

It will be appreciated that the one or more motion sensors are able to help when monitoring and diagnosing the user's neurological conditions, as they are able to provide real-time measurements of various motion signals that are associated with the user's neurological conditions. The real-time measurement may allow for earlier detection and more accurate diagnosis, which potentially leads to better treatment outcomes for the user. For example, such one or more motion sensors may be used to measure or infer, an orientation of the user's body. Notably, the orientation of the user's body is indicative of an extent of gait in the user's body. By measuring the orientation of the user's body in real-time, the one or more motion sensors are able to provide a more accurate assessment of disease progression and treatment response for the gait.

The technical effect of using the one or more motion sensors is that minute changes in the motion pattern may be sensed quickly, thereby enabling the wearable device to respond quickly. Beneficially, the one or more motion sensors may be miniaturized, thereby making them ideal for incorporation into the wearable device (for example, by making use of MEMs fabrication techniques).

Optionally, the wearable device includes at least one of: an inclinometer, a motion sensor, an accelerometer, an accelerometer that is also able to function as an inclinometer, a gyroscope, a magnetometer, all for generating a movement signal representative of a movement of the user, for example when influenced by the symptoms of the user's neurological condition. Throughout the present disclosure, the term “movement signal” refers to a signal that contains information related to movement patterns of the user, for example under an influence of the user's neurological condition. It will be appreciated that the symptoms of the neurological condition affect the movement of the user which is sensed and represented in the movement signal. Throughout the present disclosure, the term “motion sensor” refers to a sensor that is able to sense the movement of the user. Throughout the present disclosure, the term “accelerometer” refers to a sensor that is able to sense acceleration and change in movement velocity of the user. Throughout the present disclosure, the term “gyroscope” refers to a sensor that is able to sense an angular orientation and angular velocity of the movement of the user. Throughout the present disclosure, the term “inclinometer” refers to a sensor that is able to sense an inclining angle of a part of the user (for example, the user's sternum) relative to the direction of gravitational force (namely a vertical direction). The technical effect of including the motion sensor, at least one of: an inclinometer, a motion sensor, an accelerometer, a gyroscope, is that the wearable device is able to accurately and precisely generate the movement signal. The movement signal is included in the input signal referred to in the present disclosure. Throughout the present disclosure, the term “accelerometer” refers to the sensor that can measure acceleration in the X, Y, and Z axes and which can detect a deviation from the vertical alignment. When the accelerometer is oriented vertically along the user's body, most of the gravitational force is along the Y-axis with minimal deviations in the X and Z axes. Any deviations from the upright posture of the user increase the gravitational component along the X-axis or Z-axis and decrease it along the Y-axis. Throughout the present disclosure, the term “gyroscope” refers to the sensor that can measure an angular velocity. By continuously monitoring the angular velocity of the user's body, the gyroscope can detect deviations from the user's gait pattern. The technical effect of including the motion sensor in the wearable device is the possibility of detecting the inclination angle of the upper torso (the angle at which the upper part of the body deviates from an upright posture of the user) as well as the possibility of monitoring the dynamic status of gait and body. Accurate detection of deviations from the upright posture or gait of the user is crucial for tracking the progression of neurological disorders and treatment efficiency. Optionally, the wearable device can not only detect deviations from the user's upright posture or gait but can also provide immediate feedback to the user that the deviation was detected.

Optionally, the wearable device is configured such that the sensor arrangement includes one or more biosensors and also one or more motion sensors to sense biological patterns of the user, wherein the biological patterns are associated with the symptoms of the user's neurological condition. The one or more biosensors enable to detect biological parameters or biological patterns of the user, wherein the biological patterns are associated with the symptoms of the neurological condition of the user. In this regard, a biosensor is a device that senses one or more biological parameters or biological patterns, such as the user's temperature, the user's nerve signals, the user's cardiac signals, the user's blood sugar level, the user's blood pressure, the user's heart beating rate, the user's bodily enzymes, the user's skin conductivity, the user's skin impedance, the user's skin color (for example, status of skin capillary dilation), user's blood flow Doppler measurement or antibodies, by converting the biological pattern into a corresponding electrical signal. It will be appreciated that the one or more biosensors are able to help when monitoring and diagnosing the user's neurological conditions, as they are able to provide real-time measurements of various biological signals and biomarkers that are associated with the user's neurological conditions. The real-time measurement may allow for earlier detection and more accurate diagnosis, which potentially leads to better treatment outcomes for the user. For example, such one or more biosensors may be used to measure or infer levels of neurotransmitters, such as dopamine, in the user's brain. Dopamine measurement may be made by using pulsed magnetic transcranial stimulation and then therefore monitoring brain activity using scalp electrodes after each magnetic transcranial pulse has been applied. In Parkinson's disease, the degeneration of dopaminergic neurons leads to a decrease in dopamine levels, which is associated with the characteristic motor symptoms of the disease. By measuring dopamine levels in real-time, the one or more biosensors are able to provide a more accurate assessment of disease progression and treatment response.

The technical effect of using the one or more biosensors is that minute changes in the biological pattern may be sensed quickly, thereby enabling the wearable device to respond quickly. Beneficially, the one or more biosensors may be miniaturized, thereby making them ideal for incorporation into the wearable device (for example, by making use of MEMs fabrication techniques).

Optionally, the one or more biosensors include at least one of: a temperature sensor, a skin impedance sensor, a heart rate sensor, a microphone, an electrostatic contact sensor. Beneficially, the wearable device uses two temperature sensors, wherein a first temperature sensor is placed in intimate contact with the user's skin, and a second temperature sensor is positioned away from the user's skin (for example, external to clothing worn by the user). Optionally, the temperature sensors are implemented using thermistors and/or thermocouples. In this regard, the temperature sensors measure the user's body temperature, for example relative to an external temperature, and provide information about fever, hyperthermia, hypothermia that may affect the user. For example, people with Parkinson's disease tend to show severe symptom at low temperature conditions. Thus both body temperature of the user and external temperature to the user need to be measured. In an event of neurological conditions being encountered, the user's body temperature regulation is susceptible to being disrupted. Monitoring body temperature of the user may help to detect a sign of change in biological pattern. The skin impedance sensor (for example, a “Bio-Z” impedance sensor (EDA) for measuring galvanic skin response, wherein such a sensor may be used to measure user stress level, respiration rate and so on) measures the electrical impedance and resistance of the use's skin, which may provide information about changes in skin hydration, sweating, and other physiological responses. Studies have shown that an abrupt change in skin impedance or skin conductivity may be a suitable signal to monitor, for example when the user is suffering from Parkinson's disease, that provides a good indication circa 4.2 seconds before the user is about to fall (for example, experience a FOG event).

The heart rate sensor (for example, implemented by using ECG) measures the heart rate and provides information about heart rhythm and cardiac function (for example, occurrence of arrhythmia). Typically, the changes in heart rate and heart rhythm may potentially be an indication of neurological conditions such as autonomic dysfunction, epilepsy, or migraines. Monitoring heart rate may help to provide an early detection of such neurological conditions.

The microphone senses sounds of the user and may be used to measure breathing patterns, snoring, coughing, and other respiratory or vocalizations. In Parkinson's disease, changes in voice and speech patterns may potentially occur. Monitoring vocalizations using the microphone may help detect changes in speech patterns and vocalizations, for example voice stammer. The microphone may also be used to detect the amplitude and frequency of the auditory stimulation being provided by the wearable device to the user. The microphone can pick up the sounds produced by the auditory stimulation. Therefore it is possible to determine whether the auditory stimulation is operating correctly. Mutatis mutandis, the accelerometer, may be used to monitor the vibrotactile stimulation being provided by the wearable device to the user. When the wearable device provides vibrotactile stimulation, the accelerometer can detect the corresponding vibrations and therefore it is possible to verify whether the wearable device works as expected. If the expected vibrations are not detected it might be an indicator of malfunction of the wearable device.

The electrostatic contact sensor measures the electrical charge on the user's skin and may thereby provide information about sweat production, skin hydration, and may be used to trigger an ECG measurement and/or a bio-Z skin impedance measurement. Thus, this electrostatic measurement is useful to know for determining a suitable time to commence ECG, Bio-Z, and similar monitoring, and when to use other skin contact electrodes and sensors which require contact to the user's skin in order to measure biological signals accurately. The technical effect of using the one or more biosensors is that valuable insights into the change in biological pattern associated with the user's neurological conditions may be identified, thereby enabling diagnosis and treatment thereof.

Beneficially therefore, for the wearable device, the one or more biosensors includes at least one of: skin-contacting electrodes for contacting onto the user's skin for sensing at least one of: nerve signals, skin characteristics, cardiac signals, skin conductivity.

Beneficially therefore, for the wearable device, the one or more biosensors and the one or more motion sensors include at least one of: a temperature sensor, a skin impedance sensor, a heart rate sensor, a microphone, an electrostatic skin contact sensor, an accelerometer, an inclinometer, a gyroscopic sensor.

Optionally, the control arrangement of the wearable device further includes a computing arrangement for executing at least one algorithm for processing the input signal to determine a degree of the symptoms of the neurological condition of the user. The at least one algorithm may optionally be implemented as a simple software routine (for example, a predefined deterministic routine) that merely applies pre-defined treatments; alternatively, the at least one algorithm may be implemented as a sophisticated artificial-intelligence-based software suite.

Throughout the present disclosure, the term “computing arrangement” refers to a set of components and equipment that has data processing capabilities. Optionally, the computing arrangement includes a microprocessor coupled to an associated data memory, wherein the data memory is configured to store software that, when executed, implements data processing algorithms. Throughout the present disclosure, the term “at least one algorithm” refers to a set of instructions that are executed by the computing arrangement to determine the symptoms of the neurological condition in the movement signal. Notably, the at least one algorithm determines a form and an intensity of the at least one of gait, hesitation and tremor and correspondingly adjusts the frequency, duration and amplitude of the vibrotactile stimulation and/or auditory stimulation with cueing (for example, as described in the foregoing) to be applied to the user. For example, if the gait is determined by the at least one algorithm to be severe (namely, greater than a threshold trigger value for the gait), then the amplitude and the duration of the vibrotactile stimulation and/or auditory stimulation, for example optionally also electrical stimulation, to be applied to the user is made correspondingly greater and longer, respectively. Optionally, the at least one algorithm takes into consideration a muscle fatigue occurring as a result of the applied stimulation and makes a corresponding adjustment, for example by varying the frequency and amplitude of the stimulation applied for compensating for the fatigue and thereby continuing to mitigate effectively the gait that occurs during a walking movement of the user. The technical effect of executing the at least one algorithm is that the wearable device is able to accurately determine the degree and severity of the symptoms of the user's neurological condition. Thus, advantageously, the wearable device is able to determine precise parameters of stimulation that is to be applied by the wearable device to the user for effectively mitigating the symptoms of the user's neurological condition.

Optionally, as aforementioned, the at least one algorithm is implemented as simple control software; in other words, the at least one algorithm is a defined deterministic control algorithm, namely follows simple rules that are pre-defined (in other words, a simple control algorithm that is not based on AI or machine learning). For example, Vᵢₙ(t) is the input signal in respect of time t that is provided from the sensor arrangement of the wearable device to an algorithm ALG1 executing in the control arrangement to generate intermediate data E(t) according to Equation 1 (Eq. 1):

E(t)=ALG1 (Vᵢₙ(t)) Eq. 1

The ALG1 provides an analysis of the input data to determine a status of the user.

The output signal Vₒᵤₜ(t) used to drive the stimulation arrangement is generated from the intermediate data E(t) by using a second algorithm ALG2 according to Equation 2 (Eq. 2):

Vₒᵤₜ(t)=ALG2 (E(t) Eq. 2

The algorithm ALG1 is determined from clinical trials to aggregate data from a plurality of users and identifying patterns of changes in sensor signals that give rise to different symptom states of the user, for example an onset of gait, an onset of tremor, an onset of freezing-of-gait (FoG). The algorithm ALG2 conveniently uses a look-up table relating suitable stimulation to be applied to the user when a given state of the user has been identified. Coefficients of the look-up table are beneficially updated from time-to-time, for example by downloading data from an external database such as a SaaS or PaaS server, so that the wearable device may be updated to provide “best practice” treatment to the user. The coefficients are beneficially computed by aggregating results from using a large population of the wearable devices on their respective users and identifying aggregated trends regarding certain types of stimulation that are most beneficially to be used for addressing various symptom states of the users. Optionally, the coefficients are adaptively updated as a function of time, for example as experience of the using the wearable devices grows and matures. The algorithms ALG1 and ALG2 are optionally pre-defined, and the look-up table is determined from collected data obtained using the large population of the wearable devices (for example, via clinical trials).

Alternatively, optionally, for example when the at least one algorithm is implemented as a sophisticated software suite, the at least one algorithm includes at least one: a neural network, a Hidden Markov Model, a Boltzmann machine, an adaptive neural network. The term “neural network” as used herein refers to a network of artificial neurons programmed in software, wherein the neurons are configured to function in a manner that seeks to simulate, at least in part, functioning of the human brain, for example to perceive images, video, sound, text, and so forth. The neural network typically comprises a plurality of node layers, containing an input layer, one or more intermediate hidden layers, and an output layer, interconnected, such as in a feed-forward manner (i.e. flow in one direction only, from input to output). Moreover, the neural networks are trained on large sets of data (for example, derived from clinical trials) to identify patterns and make predictions. Notably, the training dataset comprises the data obtained from a plurality of movement signals obtained from users. Optionally, training the neural networks may be performed through forward propagation (i.e. from input to output) as well as backpropagation (i.e. from output to input). Optionally, the neural network is a convolutional neural network that refers to a specialized type of neural network model developed for working with multidimensional image data such as 1D, 2D, 3D, and so forth.

The term “hidden Markov model” (HMM) as used herein refers to a Hidden Markov Model that is a statistical model that is used to model a system that is assumed to be a Markov process with hidden states. The HMM consists of two types of states: hidden states and observable states. The hidden states represent the unobserved aspects of the system being modelled, while the observable states represent the observed aspects of the system. The HMMs are widely used to analyze sequential data, such as the movement signals generated by the sensor arrangement. The hidden Markov model enables the at least one algorithm to learn the underlying structure of the sequential data and use that to make predictions about the future movements of the user.

The term “Boltzmann machine” as used herein refers to a type of artificial neural network that uses a stochastic approach to perform learning and pattern recognition. Typically, the Boltzmann machine uses a process called stochastic gradient descent to update the weights of the connections between the nodes. Optionally, the Boltzmann machine is used to learn the relationships between various characteristics of the movement signals and generate a drive signal that is tailored to correct those specific issues. The Boltzmann machine is useful for unsupervised learning tasks, such as clustering, feature extraction, and dimensionality reduction. The Boltzmann machine can also be used for supervised learning tasks, such as classification and regression, by adding an additional layer of output units.

The technical effect of implementing the aforementioned machine learning (namely, artificial intelligence) algorithms in the computing arrangement of the wearable device is to improve the accuracy of determining the degree of the symptoms of the neurological condition of the user. Beneficially, by adapting to the unique characteristics of each user, the algorithms are able to generate more effective output signals for exciting the stimulation arrangement, wherein the more effective output signals are adapted to address specific issues in the neurological condition of the user.

Optionally, the user interface arrangement is used for adjusting the at least one algorithm being executed by the computing arrangement. Notably, the user may adjust the at least one algorithm to vary the amplitude and the duration of the vibrotactile stimulation and/or auditory stimulation applied by the wearable device, according to the user's requirements. For example, the user feels that the amplitude and the duration of the vibrotactile stimulation and/or auditory stimulation determined by the at least one algorithm based on the degree of the at least one of gait, hesitation and tremor is too great in amplitude, the user is able to use the user interface arrangement to give input to reduce the amplitude and the duration of the stimulation applied to the user. The technical effect of using the user interface arrangement to adjust the at least one algorithm is that the user is able to effectively control adjustments of the parameters related to the stimulation. Beneficially, the at least one algorithm is able to learn from the user's adjustment and thereby adaptively optimize the stimulation provided by the wearable device to the user.

Optionally, the power source includes at least one rechargeable battery. In this regard, the wearable device may further comprise a charging portion (for example an electric charging portion), configured to charge the rechargeable battery. The charging portion may be configured to receive electric power via a wired connection, from an external electric power source (for example, using a pin charging method, such as USB charging or any other appropriate method). It will be appreciated that a charging portion has a charging slot with a receiving groove (USB-A connector, a USB-B connector or a USB-C connector, a POGO PIN connector) for receiving a charging head (shaped as a cylindrical pin of various sizes, a USB, or a universal AC-DC connector) of a wired connector connected on the other end to the external electric power source, wherein the receiving groove is suitably sized and shaped to accommodate a charging head of a wired connector having a size and a shape complementary to the receiving groove, such as in a lock and key arrangement.

Alternatively, the charging portion may be or comprise one or more coils or windings (such as Litz wire or Copper wire) configured to receive electric power wirelessly from an external power source (such as one or more powered coils or windings). Beneficially, the charging portion configured to receive electric power wirelessly may result in a simpler charging process, for example simply placing the wearable device within a pre-determined distance of an external electric power source. Additionally, the charging portion configured to receive electric power wirelessly may further improve ease of operation for users suffering from freeze of gait, tremors, slowness or stiffness. Such a charging portion may remove the need for a wired charging connector to be physically connected to a charging port in the wearable device in order to charge the wearable device.

Optionally, the computing arrangement is configured to be coupled via a communication arrangement to at least one of:
(i) another wearable device to transfer operating parameters such as user settings to the another wearable device, so that the wearable device and the another wearable device may be used alternately by the user (without needing to input parameters each time when switching between devices);
(ii) a remote database or server arrangement providing a software-as-a-service (Saas) or platform-as-a-service (PaaS) for providing for at least one of: updating software of the computing arrangement for operating the wearable device (for example, for updating the aforesaid at least one algorithm), for recording a record of treatment provided by the wearable device to the user, for providing remote monitoring of operation of the wearable device;
(ii) to other such wearable devices (for example, to synchronize occurrence of their respective cueing periods). Such synchronization, when invoked, is beneficially performed periodically to ensure that the wearable devices remain suitably synchronized. In this regard, the term “communication arrangement” refers to an arrangement of components that enables the wearable device to communicate and share information with the another wearable device and/or the remote database or the server arrangement (for example, the SaaS or PaaS server, as aforementioned). Throughout the present disclosure, the term “another wearable device” refers to another of the wearable device according to any of the embodiments of the present disclosure that is used simultaneously along with the wearable device by the user. The technical effect of the transfer of the operating parameters to the another wearable device is that the user is able to conveniently use the another wearable device at the desired user setting which were previously adjusted for the wearable device without the need for readjusting the another wearable device. Throughout the present disclosure, the term “SaaS” refers to the remote database or the server arrangement acting as a third-party provider for hosting software applications. Throughout the present disclosure, the term “PaaS” refers to a cloud computing model in which a third-party provider provides a platform for users to develop, run and manage their applications. The technical effect of communicating with the remote server or the remote database is that the wearable device is able to regularly update the algorithms executed by the computing arrangement. Moreover, the wearable device is able to use the record of the treatment provided by the wearable device in the past to readjust the operating parameters in order to provide more effective treatment to the user. Advantageously, any third person such as a doctor or family member of the user is able to remotely monitor the operation of the wearable device.

Optionally, the wearable device further includes a housing for accommodating the power source, the simulation arrangement and the control arrangement. Optionally, the housing may be of or comprise a plastics material (for example, polypropylene or polycarbonate) or a metal or alloy of metal (for example, aluminium, preferably anodized aluminium); optionally, the housing may be manufactured from recycled plastics material to render the wearable device more environmentally friendly in manufacture. Beneficially, the aluminium housing is lightweight, extremely strong, naturally protects electronics, 100% recyclable and non-toxic material and reducing noise by serving as an excellent sound absorber to sound produced within the wearable device; optionally the aluminium housing is manufactured from recycled aluminium. The housing may protect the dissipating portion from impact, scratches or other degradation which could affect performance of the dissipating portion. Optionally, the washable housing is further accommodated in a washable covering, wherein the covering is of suitably shape, size and durability to accommodate at least partially the housing with all of the components of the wearable device therein. It will be appreciated that the covering, like the housing, leaves the surface configured to deliver the vibrotactile stimulation to the user exposed. Thus, in summary, the control arrangement is configured to process the input signal (Vᵢₙ(t)) and to generate the corresponding output signal (Vₒᵤₜ(t)) to excite the stimulation arrangement to generate the vibrotactile stimulation within a frequency range of 20 Hz to 500 Hz during the pulsing period, optionally within a frequency range of 30 Hz to 300 Hz, optionally within a frequency range of 80 to 250 Hz, more optionally in a range of 80 Hz to 200 Hz. The most preferable range is the range of 80 Hz to 250 Hz. It was found that the upper limit 250 Hz is the most sensitive frequency for vibrotactile stimulation, in the sense that the human skin is particularly receptive to vibrations at this frequency allowing the users to perceive vibrations more intensely compared to other vibrations. Optionally, the control arrangement is configured to implement the cueing period (VP_{cueing}) to have a duration in a range of 0.2 seconds to 5 seconds, optionally a duration in a range of 0.2 seconds to 4 seconds. Optionally, the stimulation arrangement is configured to apply the vibrotactile stimulation during the pulsing period (VPₚᵤₗₛₑ) to have a frequency that is independently adjustable to an amplitude of the vibrotactile stimulation, as aforementioned.

Optionally, the mounting arrangement includes a strap arrangement for use when securing the wearable device to at least one region of the user, including: a neck region of the user, a chest region of the user, a sternum region of the user, a limb region of the user, a hip region of the user, a wrist region of the user, a head region of the user. The term “strap arrangement” as used herein refers to an arrangement of one or more straps that are designed for securing a wearable device on a user's body by encircling or securing the one or more straps of the arrangement around the user's body. Herein, the user refers to an individual (namely, a human, a patient, a subject, and so forth) who utilizes the strap arrangement with the wearable device for at least one of: therapeutic purposes, diagnostic purposes and monitoring purposes. It will be appreciated that the structure of the strap arrangement is designed to provide a secure and comfortable fit to the user, while also allowing for easy adjustment and removal of the wearable device as needed. Optionally, the one or more straps may have various configurations depending on the intended use. For example, the configurations may include crossed or diagonal straps, vertical or horizontal straps, or a combination of both.

Optionally, the wearable device may be placed against any body part such as a sternum, arm, shoulder, wrist, neck, ankle, leg, hip and temple or head region of the user. It will be appreciated that the aforementioned body parts allow for easy access of the wearable device by the user and by a carer or family member of an incapacitated user. The term “at least one region” as used herein refers to one or more areas of the user's body where the wearable device is secured for optimal therapeutic benefits. In this regard, the at least one region includes the neck region, the chest region, the sternum region, the limb region, the hip region, the wrist region and the head region of the user. Moreover, the strap arrangement is configured to secure the wearable device to one or more of the aforementioned regions, depending on the specific needs of the user and the intended application of the wearable device. For example, if the wearable device is intended to monitor tremors in the user's head, the strap arrangement may be configured to secure the wearable device to the user's head region. In such a case, the strap arrangement is wrapped around the user's head for securing the wearable device in place. Herein, the term “sternum region” refers to an anatomical location substantially at a center of a chest of a human. Notably, the sternum region is an attachment point for several major muscle groups that involve pectoralis muscles, abdominal muscles, muscles of neck and shoulders. Thus, by applying the vibrational stimulation to the sternum region of the front chest region, the wearable device is able to effectively control the stability of the upper body of the user. Optionally, the major parameters of vibrotactile stimulation such as vibrotactile stimulation frequency, vibrotactile stimulation amplitude, cueing period (or cueing frequency) and pulsing period, are susceptible to being adjusted or modulated to reduce the symptoms of Parkinson's disease.

Optionally, the strap arrangement is manufactured from at least one of: woven cloth, flexible tape, cotton cloth, polyester cloth, polypropylene cloth, Kevlar® cloth, Carbon fibre cloth, polyamide cloth, silk cloth, linen cloth. The term “woven cloth” as used herein refers to a fabric made by weaving two or more sets of yarn or thread together at right angles to form a stable, durable material. Optionally, the woven cloth is made from a variety of natural and synthetic fibers, such as cotton, polyester, or nylon®. The term “flexible tape” as used herein refers to a thin strip of material that may be easily bent or moulded to conform to the shape of the user's body. Optionally, the flexible tape is made from a variety of materials, such as plastics materials, textile fabric, or rubber. The term “cotton cloth” as used herein refers to a natural fiber that is a soft and breathable fabric. Typically, the cotton cloth is hypoallergenic and comfortable to wear. The term “polyester cloth” as used herein refers to a synthetic fabric that is durable, lightweight, and resistant to wrinkles and shrinkage. The term “polypropylene cloth” as used herein refers to a synthetic fabric that is lightweight, durable, and resistant to water and chemicals. The term “Kevlar® cloth” as used herein refers to a registered trademark of DuPont and is a strong, lightweight material. The term “Carbon fiber cloth” as used herein refers to a strong, lightweight material that is made from woven strands of Carbon atoms. The term “Polyamide cloth” as used herein refers to a synthetic fabric that is strong, lightweight, and resistant to abrasion and chemicals. The term “silk cloth” as used herein refers to a soft natural fiber that is hypoallergenic and breathable. The term “linen cloth” as used herein refers to a strong natural fiber, durable fabric that is breathable and absorbent.

The technical effect of manufacturing the strap arrangement using the aforementioned materials is that they are lightweight and flexible, thus making them comfortable to wear for extended periods of time. Moreover, the aforementioned materials are strong and durable, ensuring that the strap arrangement is able to withstand repeated use and wear. Furthermore, the aforementioned materials are hypoallergenic and non-irritating, reducing the risk of skin irritation or allergic reactions in sensitive users.

Thus, in summary, the control arrangement is configured to generate the output signal to excite the stimulation arrangement to generate the vibrotactile stimulation within a frequency range of 20 Hz to 500 Hz during the pulsing period, optionally within a frequency range of 30 Hz to 300 Hz, optionally in the range of 80 Hz to 250 Hz, more optionally in a range of 80 Hz to 200 Hz. Optionally, the control arrangement is configured to implement the cueing period (VP_{cueing}) to have a duration in a range of 0.2 seconds to 5 seconds, optionally a duration in a range of 0.2 seconds to 4 seconds. Optionally, the stimulation arrangement is configured to apply the vibrotactile stimulation during the pulsing period (VPₚᵤₗₛₑ) to have a frequency that is independently adjustable to an amplitude of the vibrotactile stimulation, as aforementioned.

Thus, in summary, the stimulation arrangement includes at least one actuator, for example just a single actuator, for example two or more actuators, that is vibrationally coupled when in use to a skin region of the user. Optionally, the at least one actuator includes at least one of: a linear resonant actuator, an eccentric rotating mass (ERM) DC motor, an eccentric rotating mass brushless (ERM BLDC) motor, a piezo-electric actuator. More optionally, the at least one actuator includes a plurality of linear resonant actuators whose resonant frequencies are mutually different, to provide the stimulation arrangement in aggregate with an extended frequency operating range when generating vibrotactile stimulation.

Optionally, the wearable device further includes an alarm arrangement that is configured to provide a stimulation alarm alert in an event of a condition arising needing the user's attention, wherein the stimulation alarm alert is provided by at least one of: a vibrotactile stimulation alarm alert, an auditory stimulation alarm alert. Throughout the present disclosure, the term “alarm arrangement” refers to an arrangement of components that are capable of occasionally alerting the user in case of a possible emergency that requires an immediate attention of the user. Notably, the attention of the user is gained by providing the stimulation alarm alert via the alarm arrangement in which a certain type of stimulation is provided to the user to act as the alarm. Herein, the vibrotactile stimulation alarm alert optionally refers to using vibrotactile stimulation as the alarm for alerting the user. Likewise, the auditory stimulation alarm alert refers to stimulating the user with an audio alarm to alert the user. Optionally, the stimulation alarm alert is provided to user in case of an incoming series of the symptoms of the neurological condition in order to prepare the user in advance. Alternatively, the stimulation alarm alert is provided to the user in a case of malfunctioning in the wearable device so the user can act accordingly to prevent any health emergency from occurring. The technical effect is that an added protection is provided to the user in case of emergency scenarios.

Optionally, the wearable device further includes a user interface arrangement that is configured to be used by the user for adjusting operating parameters of the wearable device, wherein the operating parameters include at least one of: a duration (VPₚᵤₗₛₑ) of the pulsing period for vibrotactile stimulation, a duration (VPᵣₑₛₜ) of the resting period for vibrotactile stimulation, a duration (VP_{cueing}) of the cueing period, wherein the duration (VP_{cueing}) of the cueing period is a sum of the duration of (VPₚᵤₗₛₑ) of the pulsing period and the duration (VPrest) of the resting period an amplitude (VS2) of the vibrotactile stimulation applied to the user during the pulsing period for vibrotactile stimulation, a frequency of the vibrotactile stimulation, a duration (APₚᵤₗₛₑ) of the pulsing period for auditory stimulation, a duration (APᵣₑₛₜ) of the resting period of auditory stimulation, an amplitude (AS2) of the auditory stimulation during the pulsing period for auditory stimulation, whether or not to provide a vibrotactile stimulation alarm alert and/or an auditory stimulation alarm alert during the pulsing period or prior thereto.

Optionally, the wearable device is configured to function in a mode selected from any of:
(i) a sensing-only mode, wherein the wearable device is configured to use the sensor arrangement to sense motion patterns and/or biological patterns of the user and disabled from providing vibrotactile stimulation and/or auditory stimulation;
(ii) an acting-only mode, wherein the wearable device is configured to provide vibrotactile stimulation and/or auditory stimulation with cueing according to a defined pattern and not in response to information provided by the sensor arrangement;
(iii) a sensing-acting mode, wherein the wearable device is configured to use the sensor arrangement to sense movement and/or biological parameters of the user in the input data, and to process the input data in the control arrangement to provide corresponding output data to drive the stimulation arrangement to apply stimulation to the user;
(iv) a manner mode, wherein the wearable device is user-adjustable to reduce an amplitude of the vibrotactile stimulation and/or the auditory stimulation for a temporary duration; and
(v) a synchronization mode, wherein a plurality of the wearable devices function to synchronize their respective cueing periods. Such synchronization is beneficially performed continuously or periodically by the plurality of wearable devices.

In addition the sensing-only mode, the acting-only mode and the sensing-acting mode can be selectable one out of three (only one can be active at a time), wherein the manner-mode and the synchronization mode are independent from the sensing-only mode, the acting-only mode and the sensing-acting mode. In other words the manner-mode and the synchronization mode can be used simultaneously with the sensing-only mode, the acting-only mode and the sensing-acting mode without affecting their operation.

Thus, optionally, the wearable device is configured to function in a mode selected from any of: a sensing-only mode, an acting-only mode, a sensing-acting mode, a manner mode, and a synchronization mode.

In the sensing-only mode, the wearable device may sense the user's symptom status without providing vibrotactile stimulation and/or auditory stimulation, to assist to analyse motion patterns compared to a normal user and find an appropriate cueing frequency, pulsing-period stimulation amplitude, and so forth to use. Moreover, the wearable device may analyse motion and biological patterns to determine the level of symptoms and identify a proper stimulation amplitude, frequency and duration.

In acting-only mode, the wearable device provides vibrotactile stimulation and/or auditory stimulation, but does not sense motion and biological patterns of the user.

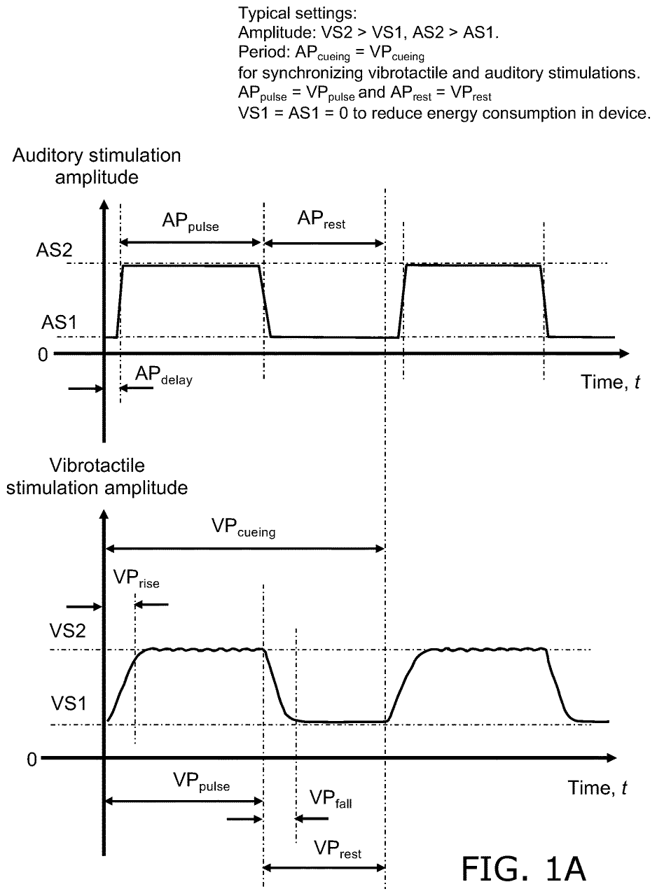

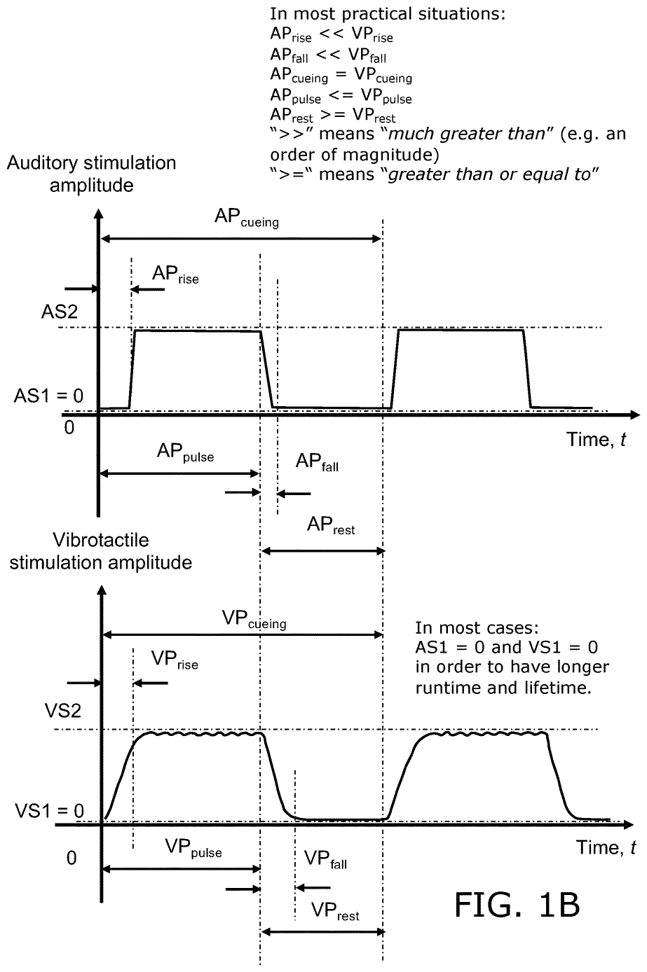

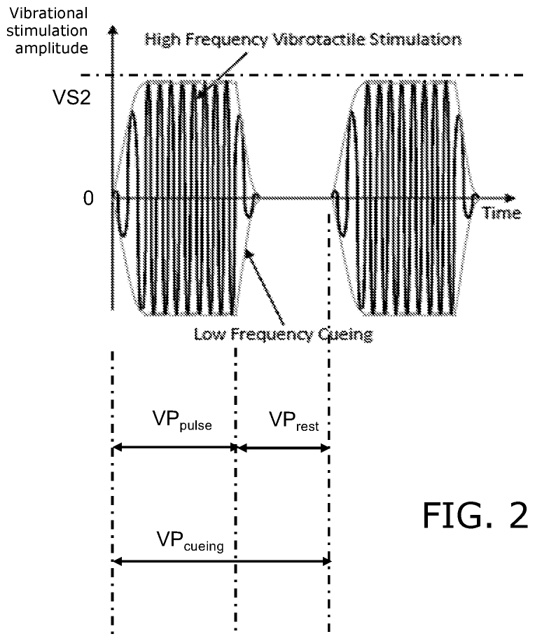

In sensing-acting mode, the wearable device provides vibrotactile stimulation and/or auditory stimulation during the cueing period, for example as illustrated in FIGS. 1 and 2. Additionally, the wearable device determines motion and biological patterns by using at least one algorithm by analysing the known vibration and sound patterns from the wearable device and thus the motion and biological patterns may be isolated and analysed separately. In addition, in the sensing-acting mode the wearable device can use a closed-loop feedback control, which can operate in two ways: real-time and non-real-time. Optionally in a non-real-time closed-loop feedback control process the wearable device is configured to collect data including stimulation settings, log data and sensor data and to store this information in a memory (e.g flash memory). The collected data is sent periodically to a server arrangement (e.g a cloud server). The server arrangement is configured to analyse the collected data, severity of symptoms, a relationship between stimulation settings and the severity of symptoms. Optionally the collected data is analysed by the server arrangement by means of big data and/or AI algorithms. Therefore, by collecting the data and analysing it is possible to achieve the advantageous technical effect of providing optimal stimulation settings tailored to the individual user's needs.

In the manner mode, by using the button of the user interface arrangement, the user is able to adjust the vibrotactile stimulation amplitude and/or auditory stimulation amplitude provided by the wearable device.

In the synchronization mode, the user is using several wearable devices on different body parts at the same time; in such a case, the wearable devices are beneficially configured to be mutually synchronized in terms of one or more of their vibrotactile stimulation frequency, vibrotactile stimulation amplitude, duration of the cueing period, duration of the pulsing period, duration of the resting period. The user interface arrangement may optionally help the user synchronize all the wearable devices.

Thus, in summary, the control arrangement includes a computing arrangement for executing at least one algorithm for processing the input signal to determine a degree of the symptoms of the neurological condition of the user. Optionally, the at least one algorithm includes at least one: a pre-defined deterministic algorithm, a neural network, a Hidden Markov Model, a Boltzmann machine, an adaptive neural network, a Fast Fourier Transform (FFT)-based algorithm, a Support Vector Machine (SVM) algorithm.

Optionally, the aforesaid user interface arrangement is configured to be used for adaptively adjusting the at least one algorithm being executed by the control arrangement.

Optionally, the control arrangement is configured to be coupled via a communication arrangement to at least one of:
(i) another wearable device to transfer operating parameters such as user settings to the another wearable device, so that the wearable device and the another wearable device may be used alternately by the user;
(ii) a remote database or server arrangement providing a software-as-a-service (Saas) or platform-as-a-service (PaaS) for providing for at least one of: updating software of the control arrangement for operating the wearable device, for recording a record of treatment provided by the wearable device to the user, for providing remote monitoring of operation of the wearable device; and
(iii) to other such wearable devices (for example, to mutually synchronize occurrence of their respective cueing periods).

Optionally, the computing hardware of the wearable device is configured to use a filter to filter the input data received from the sensing arrangement to remove interference and noise therefrom to generate corresponding filtered input data; conveniently, such filtering may be achieved by using at least one of:
(i) digital recursive filters, wherein the recursive filters are optionally adaptable to attenuate specific types of interference and noise present in the input data;
(ii) Fast Fourier Transform filters for converting the input data (Vᵢₙ(t)) via a Fast Fourier Transform (FFT) algorithm to generate corresponding spectral Fourier coefficients, and then removing contributions from specific spectral Fourier coefficients corresponding to interference and noise; the remaining spectral Fourier coefficients after removal of the spectral Fourier coefficients corresponding to noise and interference are then used in further computations executed by the computing hardware; and
(iii) Hidden Markov Models (HMM) filters that are configured to apply a signal processing model to separate desired components of the input data from noise and interference present in the input data; and then generating the filtered input data from the desired components.

The digital recursive filters, Fast Fourier Transform filters, and Hidden Markov Models are beneficially implemented as algorithms that are implemented in software routines that are executable on the computing hardware. As aforesaid, the filtered input data is provided to other algorithms implemented in executable software running on the computing hardware. When the wearable device employs vibrotactile stimulation, it generates vibrations that can interfere with the motion and/or biosensor reading. However, the wearable device used in conjunction with the software product is capable of logging detailed user data, including each interaction with the device, setting adjustments, and vibrotactile stimulation parameters. Therefore, the mentioned parameters become predictable parameters within the data stream and can be distinguished from motion sensors and/or biosensor readings. The technical effect associated with the incorporation of filtering algorithms together with data gathering is the possibility of effective separation of the biosignal and/or motion signal of the user from the vibrations caused by the wearable device itself. Therefore, it is possible to provide more accurate and reliable readings of the user's biosignal patterns and/or motion patterns. Optionally, the mounting arrangement of the wearable device is configured for securing the wearable device to at least one region of the user, including: a neck region of the user, a chest region of the user, a sternum region of the user, a limb region of the user, a hip region of the user, a wrist region of the user, a head region of the user. Optionally, the mounting arrangement includes a strap arrangement that is manufactured from at least one of: woven cloth, flexible tape, cotton cloth, polyester cloth, polypropylene cloth, Kevlar® cloth, Carbon fibre cloth, polyamide cloth, silk cloth, linen cloth. Optionally, the strap arrangement is adjustable to the user by using at least one of: adhesive fasteners, magnetic fasteners, Velcro®-type fasteners, pop-rivet fasteners, buckle fasteners, cloth clamps. The term “adhesive fasteners” as used herein refers to fasteners that use an adhesive material to attach the strap to the user's body. The term “magnetic fasteners” as used herein refers to fasteners that use magnets to secure the strap at an appropriate region of the user's body. The term “Velcro®-type fasteners” as used herein refers to a type of hook-and-loop fastener that is commonly used in clothing. The term “Pop-rivet fasteners” as used herein refers to a type of mechanical fastener that uses a rivet to attach two pieces of material together. The term “Buckle fasteners” as used herein refers to a type of fastener that uses a buckle to adjust the length of the strap. The term “Cloth clamps” as used herein refers to a type of fastener that uses a clamp to adjust the size of the strap. In this regard, the strap arrangement possesses an ability to be customized to fit the user's body, regardless of their size or shape, by using one or more of the above-mentioned fasteners or clamps. The technical effect of using the aforementioned adjustable fasteners along with the strap arrangement is that it allows precise positioning and snug fit of the wearable device, that is necessary for optimal therapeutic benefits.

According to a second aspect, there is provided a method for operating a wearable device for mitigating symptoms of a neurological condition of a user, wherein the method includes:
using a mounting arrangement for detachably mounting the wearable device to the user;
using a power source for providing electrical power to the wearable device when in use; and
using a stimulation arrangement for applying stimulation to the user;
using a control arrangement for generating an output signal for driving the stimulation arrangement for applying the stimulation to the user, wherein the wearable device is configured to excite the stimulation arrangement to provide pulsing period stimulation (VS2, AS2) during a pulsing period and to provide resting period stimulation (VS1, AS1) during a resting period, wherein the pulsing period stimulation (VS2, AS2) is greater in amplitude than the resting period stimulation (VS1, AS1), wherein the pulsing period and the resting period are included within a cueing period, wherein the wearable device is configured to provide a concatenated series of such cueing periods, and wherein the wearable device is configured to provide cueing. “Cueing” is defined in the foregoing.

Optionally, the amplitude AS1 is made to be zero (namely 0), to conserve power (namely, to reduce power consumption) within the wearable device, and also to maximize a cueing effect obtained from the wearable device. Likewise, optionally, the amplitude VS1 is made to be zero (namely 0), to conserve power within the wearable device.

In this regard, the method enables providing a combination of the pulsing stimulation and resting stimulation with cueing to the user that helps to mitigate physical symptoms associated with any of his/her medical conditions; the stimulation is beneficially applied in a focused manner, namely to a specific portion of the user's body; the stimulation is beneficially adapted or substantially synchronized to a manner of movement of the user. Thus, there is provided “focused stimulation” which means that the stimulation is applied to a local part of the user's body, in contradistinction to whole body stimulation. Moreover, the method enables altering variables of the stimulation to refine the pattern, amplitude and frequency from which the user experiences greatest benefits. Furthermore, the method serves as an effective alternative to conventional invasive or therapeutic treatments of addressing neurological conditions and other conditions such as multiple sclerosis, injury, and so forth. The method optionally enables recording and analysing the overall health (quality of life (QoL)) of the user.

According to a third aspect, there is provided a software product that is executable on computing hardware of the control arrangement of the wearable device of the first aspect, to implement the method of the second aspect.

The present disclosure also relates to the method as described above. Various embodiments and variants disclosed above, with respect to the aforementioned wearable device, apply mutatis mutandis to the method. The present disclosure also relates to the software product as described above. Various embodiments and variants disclosed above, with respect to the aforementioned wearable device and the aforementioned method, apply mutatis mutandis to the software product.

### DETAILED DESCRIPTION OF THE DRAWINGS

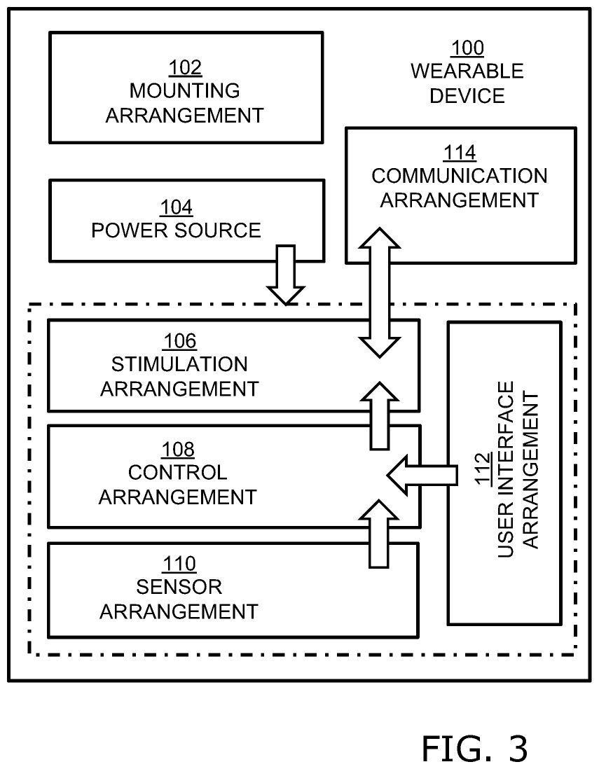

Referring to FIGS. 1A, 1B and 2, the present disclosure concerns a wearable device, denoted by 100 in FIG. 3. The wearable device 100 is configured to provide various forms of stimulation to a user, to assist the user by mitigating symptoms of a neurological condition of the user. The stimulation is provided during a cueing period having a duration VP_{cueing}. The cueing period is repeated to provide a concatenated series of such cueing periods. The series has an aggregate time duration that is a function of a dose of treatment that is required to be provided to the user. Moreover, the duration VP_{cueing} of the cueing period is optionally in a range of 0.2 seconds to 5 seconds; in view of (frequency f)=(1/period p), the wearable device 100 operates with a cueing frequency in a range of 0.2 Hz to 5 Hz. This cueing frequency is beneficially substantially synchronized with a manner of the movement of the user, for example a pace of walking of the user, a frequency of tremor of the user, a frequency of stutter or stammer of the user, a frequency of involuntary shaking of the user, and so forth; in other words, embodiments of the present disclosure use “cueing”, wherein the stimulation is made dependent upon a manner of movement of the user. During each cueing period of the duration VP_{cueing}, there is included a pulsing period having a duration VPₚᵤₗₛₑ, wherein the duration VPₚᵤₗₛₑ is in a range of 0 seconds to VP_{cueing}. During each cueing period of the duration VP_{cueing}, there is included a resting period having a duration of VPᵣₑₛₜ, wherein the resting period VPᵣₑₛₜ has a duration in a range of 0 seconds to VP_{cueing}. Moreover, a sum of the pulsing duration VPₚᵤₗₛₑ and the resting duration VPᵣₑₛₜ is equal to the cueing period VP_{cueing}, as illustrated in FIGS. 1A and 1B.

During the resting period having the duration VPᵣₑₛₜ, the wearable apparatus 100 is configured to provide a resting-period vibrotactile stimulation to the user, wherein the resting-period vibrotactile stimulation has an amplitude VS1; optionally, the amplitude VS1 is beneficially of zero magnitude (VS1=0) to maximize the cueing effect provided to the user and to save energy consumption within the wearable device 100, in most cases. Optionally, the resting-period vibrotactile stimulation is also sustained through at least a portion of the pulsing period having the duration VPₚᵤₗₛₑ. The amplitude VS1 of the resting-period vibrotactile stimulation is less than an amplitude VS2 of pulsing-period vibrotactile stimulation (namely VS2>VS1) applied during the pulsing period having the duration VPₚᵤₗₛₑ. Optionally, during the pulsing period, the pulsing-period vibrotactile stimulation is in a frequency range of 20 Hz to 500 Hz, optionally in a range of 30 Hz to 300 Hz, more optionally in a range of 80 Hz to 200 Hz although other frequency ranges are feasible to use; as aforementioned, optionally, the resting-period vibrotactile stimulation may also be applied during at least a portion of the pulsing period, such that a combination of the resting-period vibrotactile stimulation in a frequency range of 0.2 Hz to 5 Hz and the pulsing-period vibrotactile stimulation in a frequency range of 20 Hz to 500 Hz may be used, for example 30 Hz to 300 Hz, for example 80 Hz to 200 Hz. Optionally, the pulsing-period vibrotactile stimulation has an amplitude that is circa at least an order magnitude greater than an amplitude of the resting-period vibrotactile stimulation; optionally, the resting-period vibrotactile stimulation is arranged to have substantially zero magnitude (VS1=0), as aforementioned.

As illustrated in FIGS. 1A and 1B, it is often not feasible, depending on a manner in which the wearable device 100 in constructed, to attain the amplitude VS2 instantaneously, wherein a rise period having a rise duration VPᵣᵢₛₑ and a fall period having a fall duration VP_{fall} are encountered in practice. Certain types of rotating eccentric-mass vibrotactile stimulation actuators require time to angularly accelerate their respective eccentric masses, whereas multilayer piezo-electric vibrotactile stimulation actuators may respond almost instantly.

The wearable device 100 is configurable to provide auditory stimulation in addition to, or as an alternative to, the vibrotactile stimulation. As shown in FIGS. 1A and 1B, the auditory stimulation is provided during an auditory pulsing period having a duration AP_{pulse,}wherein the auditory pulsing period occurs during the cueing period having the duration VP_{cueing}. During the auditory pulsing period, there is provided auditory stimulation having an auditory stimulation amplitude AS2. There is also provided an auditory resting period after the auditory pulsing period, wherein the auditory resting period has a duration APᵣₑₛₜ. During the auditory resting period, the auditory stimulation has an amplitude AS1. Optionally, both the auditory stimulation amplitude AS1 and the vibrotactile stimulation amplitude VS1 are of a zero magnitude to provide a long run-time, based on energy stored in the wearable device 100; in other words, in most cases AS1=0 and VS1=0. The auditory stimulation amplitude AS2 is therefore beneficially, optionally, at least an order of magnitude (for example, two orders of magnitude, three orders of magnitude) greater than the auditory stimulation amplitude AS1. The auditory stimulation pulsing period may be delayed by a duration AP_{delay} from the beginning of the cueing period, as illustrated in FIG. 1. A sum of the durations AP_{delay}, APₚᵤₗₛₑ, and APᵣₑₛₜ is equal to the cueing period VP_{cueing}. Optionally, the auditory stimulation and the vibrotactile stimulation are applied simultaneously, for example during the pulsing period.

Optionally, the wearable device 100 may provide at least one of a vibrotactile stimulation alarm and an auditory stimulation alarm, in addition to the stimulations depicted in FIGS. 1A, 1B, from time-to-time, to alert the user in an event that the user needs to take action or to stop moving (for example, to take medication, to sit down, to stop moving) or if there is a danger that the user's symptoms are likely to become immediately worse (for example, in an event that the user is controlling machinery, for example taking a walk, for example driving a vehicle, where the symptoms could potentially result in a dangerous situation or accident arising).

Optionally, the auditory stimulation is synchronized with the vibrotactile stimulation such that the vibrotactile stimulation pulse period is temporally coincident with the auditory stimulation pulsing period. Alternatively, optionally, at least a portion of the auditory stimulation pulsing period occurs at a different time to the vibrotactile stimulation pulsing period; for example, the pulsing-period auditory stimulation may optionally commence slightly before the pulsing-period vibrotactile stimulation, or vice versa. Beneficially, the cueing period is synchronized with a walking pattern, tremor pattern, arm movement pattern or otherwise movement pattern of the user; in other words, the wearable device 100 is configured to utilize “cueing”. “Cueing” means that a movement pattern of the user is substantially synchronized or adapted to the duration of the cueing period, for example as aforementioned.

Beneficially, the auditory stimulation is provided in a frequency range of 100 Hz to 8 kHz, more optionally in a frequency range of 500 Hz to 5 kHz, and yet more optionally in a frequency range of 1 kHz to 5 kHz (namely, a frequency range in which a human auditory system is most sensitive).

Referring to FIG. 3, there is provided an illustration of a block diagram of a wearable device 100 for mitigating symptoms of a neurological condition of a user, according to an embodiment of the present disclosure. The wearable device 100 may be implemented to be mounted on various regions of the human body, for example in a sternum region, in an arm region (for example, in a wrist region), in a leg region (for example, in an ankle region). In general, a strap arrangement may be used to attach the wearable device 100 in use to a region of the human body; the strap arrangement may be implemented as a vest, as an arm band, as a wrist band, as a leg bad, as an ankle band, but not limited thereto. Such forms of attachment will be described in greater detail below.

As shown in FIG. 3, the wearable device 100 includes a mounting arrangement 102 for detachably mounting the wearable device 100 to the user. Moreover, the wearable device 100 includes a power source 104 for providing electrical power to the wearable device 100 when in use. Furthermore, the wearable device 100 includes a stimulation arrangement 106 for applying at least one of vibrotactile (namely, skin-contact vibrational) stimulation and auditory stimulation, for example both vibrotactile stimulation and auditory stimulation, to the user; Providing combination of the vibrotactile and auditory stimulation to the user may be significantly beneficial in alleviating symptoms of Parkinson's disease; it can enhance multisensory processing and improve the brain's ability to coordinate movement effectively. Combining vibrotactile and auditory stimulation helps mitigate physical symptoms like tremors and gait deviations but can also enhance neurological and physiological processes that enable motor and cognitive abilities. The stimulation arrangement 106 is excited (driven) in use by an output signal. Furthermore, the wearable device 100 includes a control arrangement 108 for generating the output signal for exciting (namely, driving) the stimulation arrangement 106 for applying the stimulation to the user. Additionally, the wearable device 100 includes a sensor arrangement 110 for measuring biological characteristics of the user, and for providing corresponding sensor data to the control arrangement 108. Optionally, the wearable device 100 also includes a user interface arrangement 112; a user of the wearable device 100 is able to input data and configuration instructions via the user interface arrangement 112 into the control arrangement 108, when the wearable device 100 is in use; for example, the user interface arrangement 112 enables the user to adjust one or more of the durations and amplitudes of stimulation as illustrated in FIG. 1. Optionally, the wearable device 100 further includes a communication arrangement 114 for use in exchanging data between the wearable device 100 and a remote computer or server; conveniently, the remote computer or server is configured to provide a software-as-a-service (SaaS) or platform-as-a-service (PaaS) functionality; conveniently, the remote computer or server is configured to communicate with other wearable devices, thereby enabling the remote computer or server to collate data from a plurality of the wearable devices 100 and to configure software executing in the control arrangement 108 of the wearable devices 100 to provide optimized best-practice treatment to the user to mitigate the user's symptoms.

When in operation, the wearable device 100 is configured to excite the stimulation arrangement 106 during the cueing period as illustrated in FIG. 1. During the cueing period, the wearable device 100 is configured to excite the stimulation arrangement 106 to deliver the pulsing-period vibrotactile stimulation having the amplitude VS2 in a range of 0.1 G to 2.0 G for example in a range of 0.8 G to 1.4 G force (wherein G is a force corresponding an acceleration of 9.8m²/sec acting on a mass of 1 kg). Optionally, the wearable device 100 is configured to cease exciting the stimulation arrangement 106 during a pausing period, for example to conserve energy stored in the power source 104. The pulsing-period vibrotactile stimulation is optionally provided in a frequency range of at least one of: 20 Hz to 500 Hz, 50 Hz to 500 Hz, 30 Hz to 300 Hz, 80 Hz to 200 Hz. Optionally, the wearable device 100 is also configured so that the stimulation arrangement 106 includes two or more electrodes that make contact with the user's skin for providing electrical stimulation to the user according to one or more output drive signals provided from the control arrangement 108.

The sensor arrangement 110 of the wearable device 100 beneficially includes various types of sensors. For example, the sensor arrangement 110 beneficially includes at least motion sensors, environmental sensors, a microphone and one or more biosensors. The motion sensors optionally include at least one of: an inclinometer, an accelerometer, an accelerometer configured also to function as an inclinometer, a gyroscope; beneficially these motion sensors are manufactured using MEMs technology. Beneficially, these motion sensors are 3-axis devices for measuring motion relative to a 3-axis Cartesian frame of reference. The motion sensors are beneficially used for sensing at least one of: a frequency and an amplitude of vibrotactile stimulation being applied to the user, a gait angle (namely, an angle of the plane of the user's skin at the user's sternum region relative to a vertical direction) when the user is walking, a tremor occurring in one or more limbs of the user, a freeze-of-gait (FoG) in movements of the user, a form of motion of the user when moving his/her body, but not limited thereto. Beneficially, the microphone may be used to measure a frequency and an amplitude of auditory stimulation being provided to the user. Such monitoring of the vibrotactile stimulation and auditory stimulation is beneficial, because it allows correct functioning of the stimulation arrangement 106 to be monitored, optionally allowing feedback adjustments to be made to optimize operation of the wearable device 100 when in use. The environmental sensors optionally include at least one of: one or more temperature sensors (for example, two temperature sensors, one of which is in intimate contact with the user's skin), an acoustic sensor such as a microphone, a humidity sensor, an ambient light sensor, an electromagnetic field sensor, a time clock. The environmental sensors are beneficially used for sensing environmental conditions that may potentially affect the user, for example for sensing environmental conditions that may cause unwanted neurological symptoms to arise that may afflict the user, for example induce symptoms of the user's neurological condition. The temperature sensors are beneficially used also to monitor an internal temperature of the wearable device. If the temperature sensor detects that the wearable device temperature exceeded a certain threshold value (e.g. the wearable device is overheating) it can trigger an alert to prevent damage of the wearable device and to ensure user's safety. The biological sensors optionally include two or more electrodes that make contact with the user's skin, for example for sensing skin electrical impedance over a frequency range, for sensing cardiological electrical signals (ECG), for sensing nerve signals, for sensing skin conductivity, for sensing skin surface sweat, for sensing skin humidity and so forth. Optionally, the biological sensors also include sensors for implementing at least one of: diabetic measurements, heart beating rate measurements, blood pressure measurements; optionally, some of these biological measurements are entered manually by the user (or a carer of the user) via the user interface arrangement 112 into the wearable device 100, or into a data network that is coupled in communication with the control arrangement 108 of the wearable device 100.

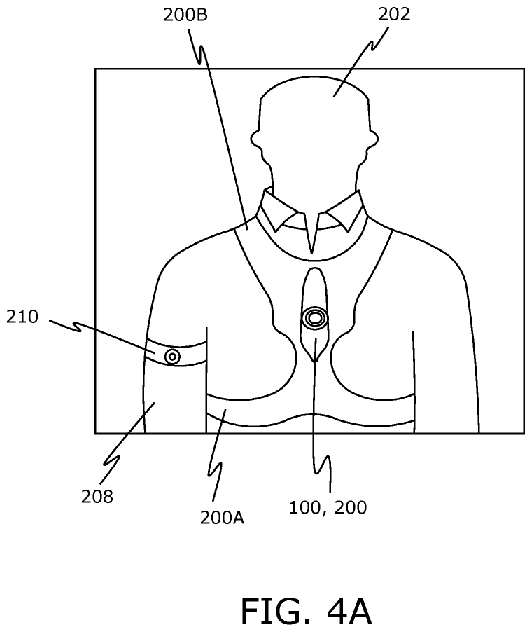

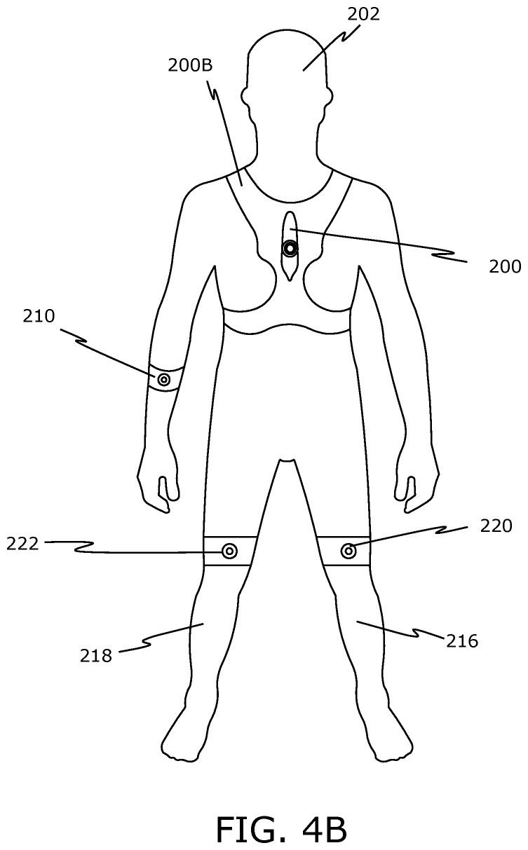

Referring next to FIGS. 4A and 4B, there are provided a front view of the wearable device 100, denoted by 200, arranged on a strap arrangement for mitigating symptoms of a neurological condition of a user 202, according to an embodiment of the present disclosure. As shown in FIG. 4A, the user 202 is shown wearing the wearable device 200 attached thereto. Optionally, the stimulation arrangement 106, denoted by 206, of the wearable device 200 is configured to apply vibrotactile stimulation to a sternum region 204 of a front chest region of the user 202. Moreover, as shown, the wearable device 200 is supported onto a vest arrangement that includes at least one strap 200A that is routed in use underneath armpits of the user 202 to encircle the sternum region 204, and further includes at least one strap 200B that is routed in use around a neck region of the user 202. As shown, a second wearable device 210 is mounted to the user 202 to encircle an arm 208 of the user 202. The second wearable device 210 is optionally mounted between the elbow and the shoulder of the user 202, or around a wrist region of the user 202. It will be appreciated that the second wearable device 210, a third wearable device 220 and a fourth wearable device 222 may be similar to, or different from, the wearable device 200. Optionally, the wearable devices 100, 210, 220 are beneficially wirelessly coupled together to be able to exchange data therebetween, for example via Bluetooth® or PLE communication protocol. Such wireless communication enables the wearable devices 100, 210, 220 to be mutually synchronized, for example in respect of their cueing periods, beneficially also pulsing periods and resting periods included in the cueing period. Beneficially, the user 202 is able to use the user interface arrangement 112 for invoking such synchronization between the multiple wearable devices 100, 210, 220, for example as aforementioned.

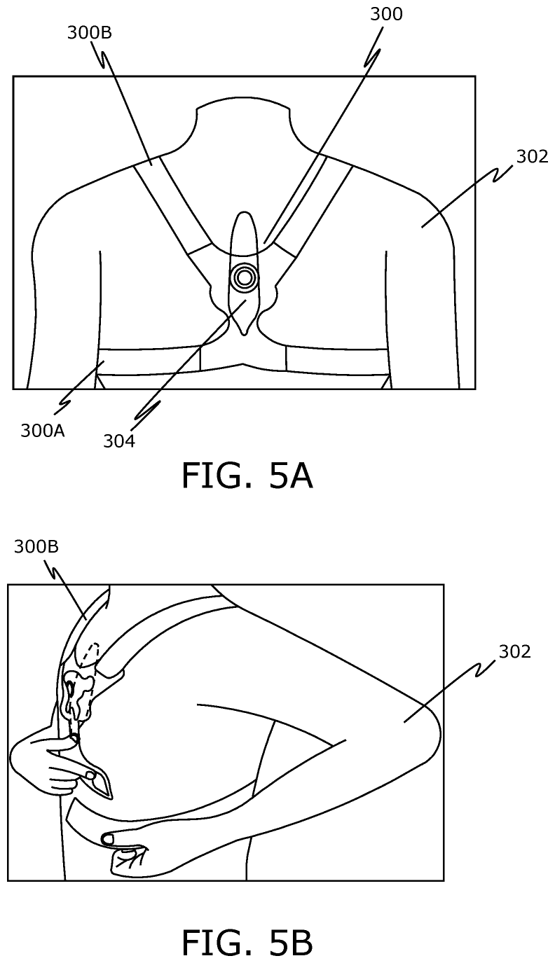

Referring to FIGS. 5A and 5B, there are provided a front view and a side view of the wearable device 100, depicted as 300, arranged on a strap arrangement for mitigating symptoms of a neurological condition of a user 302, according to an embodiment of the present disclosure. Conveniently, the wearable device 300 is attached to the strap arrangement in a repositionable manner, for example using Velcro® fasteners as described elsewhere in the present disclosure; such repositioning enables the user to find an optimal position of the wearable device 300 relative to the user's body. As shown in FIG. 5A, the front view provided is of the user 302 wearing the wearable device 300 attached thereto. Optionally, the stimulation arrangement 106 of the wearable device 300 is configured to apply vibrotactile stimulation to a sternum region 304 of a front chest region of the user 302. Moreover, as shown, the wearable device 300 is implemented as a vest arrangement that includes at least one strap 300A that is routed in use underneath armpits of the user 302 to encircle the sternum region 304, and further includes at least one strap 300B that is routed in use around a neck region of the user 302. A side view of the wearable device 300 is shown in FIG. 5B. Herein, the at least one strap 300A and the at least one strap 300B are beneficially adjusted in position on the user 302, depending on a body size of the user 302.

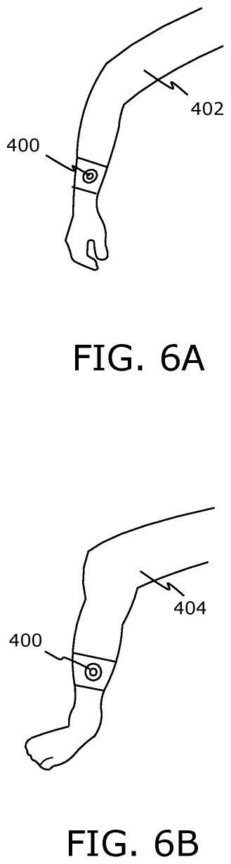

Referring next to FIGS. 6A and 6B, there are provided illustrations of an exemplary implementation of the wearable device 100, denoted as 400, arranged on a strap arrangement for mitigating symptoms of a neurological condition of a user, according to an embodiment of the present disclosure. As shown in FIG. 6A, the wearable device 400 is worn on an arm 402 of the user, for example around a wrist region of the user. As shown, the wearable device 400 is worn substantially around a wrist region of the user, although other positions are optionally feasible, for example between the shoulder and the elbow of the user to apply vibrotactile stimulation to the arm 402 of the user. As shown in FIG. 6B, the wearable device 400 is worn on a leg 404 of the user, for example around an ankle region of the user. Optionally, the wearable device 400 is worn on the user's thigh to apply vibrotactile stimulation to the leg 404 of the user.

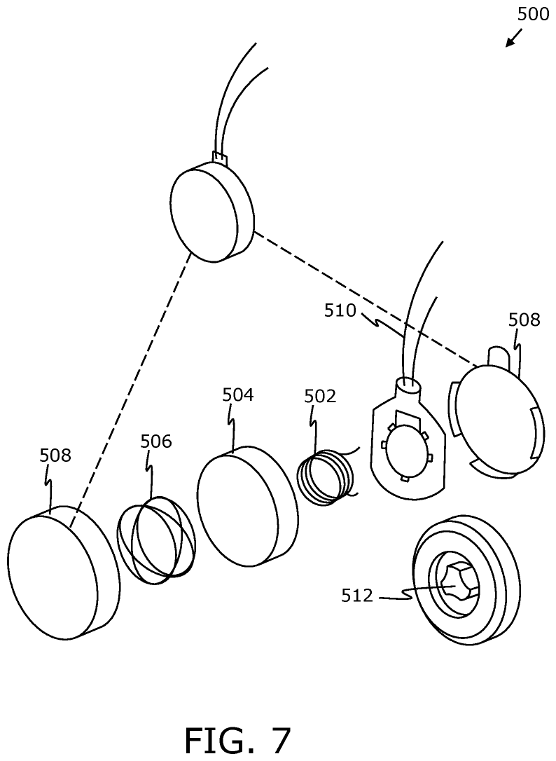

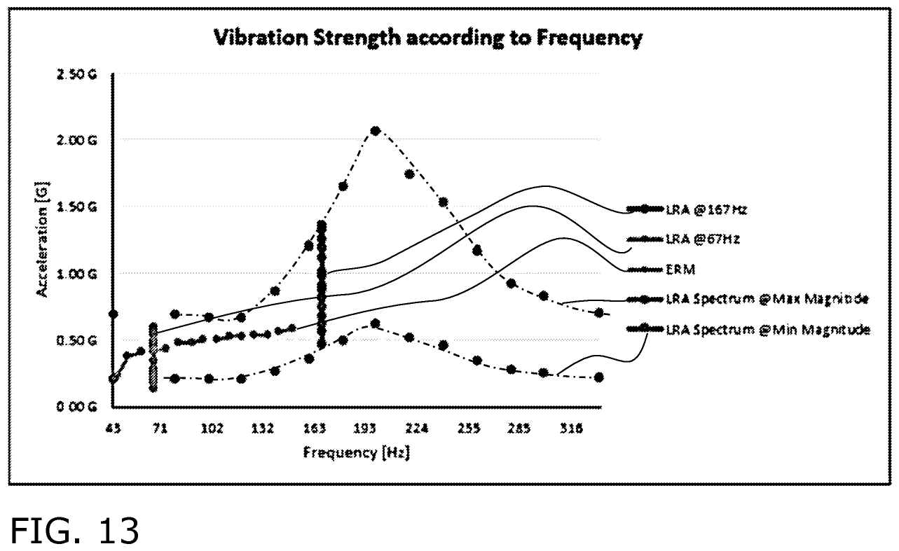

The stimulation arrangement 106 may include a variety of actuators for providing the vibrational stimulation, the auditory stimulation, or both. Optionally, combinations of mutually different actuators may be used to address different stimulation frequency ranges; for example a first vibrotactile stimulation actuator may be used to provide the pulsing-period vibrotactile stimulation during the pulsing period, for example in a frequency range of 80 Hz to 200 Hz, and a second vibrotactile stimulation actuator may be used to provide the resting-period vibrotactile stimulation during the resting period, for example in a frequency range of 0.5 Hz to 5 Hz. Moreover, it will be appreciated that the power source 104 has a modest energy storage capacity, for example when implemented as a rechargeable battery, such that the one or more actuators of the stimulation arrangement 106 are required to convert electrical power input thereto efficiently to corresponding vibrotactile stimulation to be applied to the user. Optionally, the one or more actuators for providing vibrotactile stimulation from the stimulation arrangement 106 include piezo-electric actuators (for example, multilayer piezo-electric actuators, for example manufactured from organic piezo-electric materials, alternatively Lead Zirconate Titanate (PZT)), electromagnetic actuators, but not limited thereto. To be efficient to generate the vibrotactile I stimulation for the user, the one or more actuators beneficially include respective masses that are acted upon by force relative to housings of the one or more actuators, wherein such a force may be generated electromagnetically, piezo-electrically or otherwise. It is found in practice that resonant actuators are especially beneficial to use, on account of their high efficiency of operation. For example, referring to FIG. 7, there is shown an exploded view of a linear resonant actuator 500, according to an embodiment of the present disclosure. As shown, the linear resonant actuator 500 comprises a spring-mass resonant system comprising a spring 506 coupled to a movable mass 504, wherein a solenoid coil 502 (for example implemented as a voice coil as used in a loudspeaker), is used to apply a force onto the movable mass 504 relative a housing 508 that includes electrical connections 510 for coupling to the receive an output drive signal, namely an alternating current (AC) voltage, generated in use by the control arrangement 108. In this regard, when the solenoid coil 502 is driven at the resonant frequency of a spring-mass system including the spring 506 and the mass 304, the linear resonant actuator 500 vibrates with a perceptible force, for example applying in a range of 0.1 G to 2.0 G, for example 0.5 G to 1.5 G, to a region of skin of the user. However, it will be appreciated that the linear resonator actuator 500 may optionally be implemented in alternative configurations to that illustrated in FIG. 7. It will also be appreciated that the linear resonant actuator 500 need not be excited at its resonant frequency (“natural frequency”). For example, as shown in FIG. 13, the linear resonant actuator may have a resonant frequency of substantially 200 Hz, but may be efficiently excited in a frequency range of 80 Hz to 200 Hz to provide the vibrotactile stimulation; beneficially the spring-mass system of the linear resonant actuator 500 is relatively low, for example substantially in a range of Q=3 to 6.

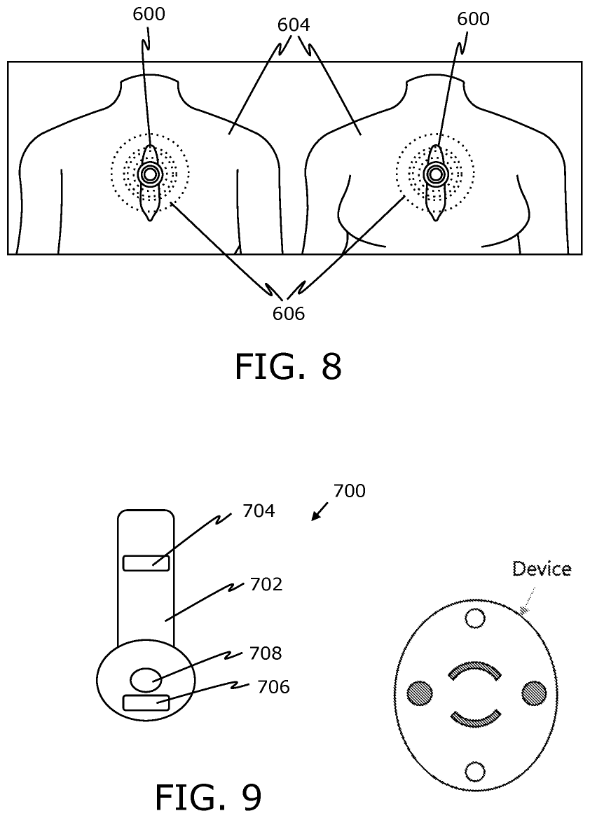

Referring to FIG. 8, there is shown a front view of a wearable device 100, denoted by 600, for mitigating symptoms of a neurological condition of a user 602, according to an embodiment of the present disclosure. In FIG. 8, there is shown a front view of a user 602 (such as a man and a woman) wearing the wearable device 600, wherein the wearable device 600 is attached to the user 602 by using an adhesive 604, for example implemented as an adhesive pad. As shown, the adhesive 604 is applied on the wearable device 600 such that the adhesive 604 does not make contact with a region of skin of the user 602. It will be appreciated that the wearable device 100, 600 may have various form factors. The wearable device 600 beneficially includes the sensor arrangement 110 to sense electrical signals accessible via contacting two or more electrodes onto the user's skin. The two or more electrodes are beneficially implemented in an adhesive pad that removably binds to the user's skin.

Referring to FIG. 8, an example form factor implementation of the wearable device 600 is shown, although it will be appreciated that other form factors for the wearable device 100, 600 are feasible pursuant to the present invention. The wearable device 600 includes an electrode arrangement that will be described in greater detail below, wherein the electrode arrangement is configured to function as bio-sensors such as bio-impedance EDG sensors and cardiac ECG sensors; the wearable device is thereby, when in use, capable of measuring the user's heart rate, and also detecting any arrhythmia in functioning of the user's heart; for example, an onset of arrhythmia may precede an onset of tremor and freezing-of-gait (FoG) in the user. From practical experience, it has been found beneficial that the sensing electrodes of the wearable device 100, 600 are mutually separated apart by a distance in a range of 2 cm to 10 cm, optionally in a range of 1 cm to 5 cm, more optionally in a range of 1 cm to 3 cm. Beneficially, the electrodes are optionally configured to be distanced slightly from where the stimulation arrangement 106 applies its vibrotactile stimulation to the user 602.

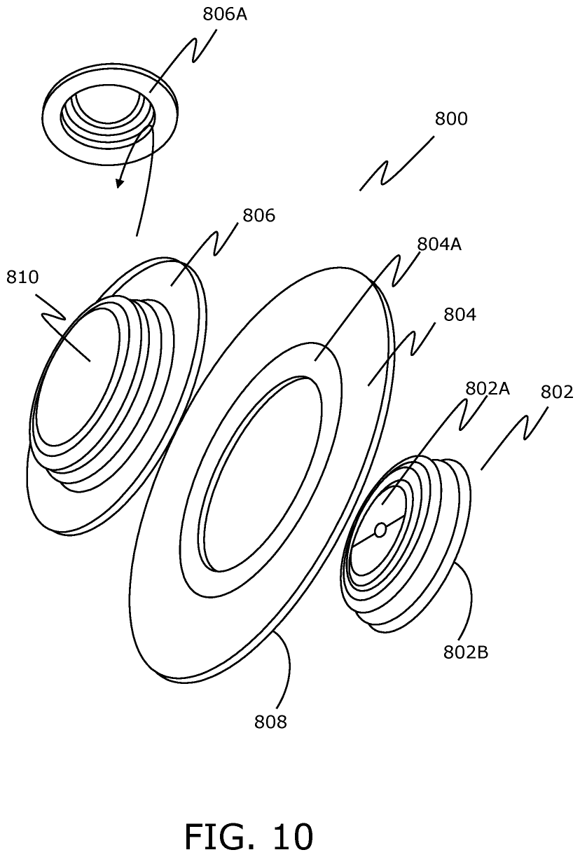

Referring to FIG. 9, the wearable device 700 comprises a device mount 702 that is a support or holder that is configured to securely attach the wearable device 700 to the user; such an attachment, namely device mount 702, may be mounted to the aforesaid strap arrangement. Optionally, a layer of cushioning memory foam is included between the device mount 702 and the wearable device 700, wherein the cushioning memory foam is compliant, meaning that it is able to conform to the contours of the skin and provide a comfortable fit to the user; the memory foam is omitted from regions of the wearable device 700, for example where the stimulation arrangement 106 makes contact with a region of skin of the user whereat vibrotactile stimulation is to be coupled; the memory foam may be conveniently manufactured from a plastics material foam, for example polyurethane foam. Moreover, the device mount 702 includes a first electrode 704 and a second electrode 706 that are configured, when the wearable device 700 is worn by the user, to sense nerve signals propagating in the user's skin, wherein the nerve signals are used as input signals to the control arrangement 108; the control arrangement 108 is configured to process the input signals and compute therefrom a suitable form of output signal for exciting the stimulation arrangement 106. The wearable device 700 includes one or more biosensors 708 that may optionally include a temperature sensor, a skin impedance sensor, a heart rate sensor, a microphone, an electrostatic contact sensor, although other types of biological sensor may optionally be used.

Referring to FIG. 10, there is shown an illustration of an exploded view of an overhead adhesive patch 800, according to an embodiment of the present disclosure. As shown, the overhead adhesive patch 800 is provided as distinct, separate layers which are individually retrievable and may be applied individually (or separately) to a wearable device 100, denoted by 802. The overhead adhesive patch 800 is conveniently implemented as a hook-and-loop fastener, such as a Velcro® fastener. The overhead adhesive patch 800 comprises a hook layer 804 and a loop layer 806. The hook layer 804 and the loop layer 806 are configured to couple to each by using tiny hooks 804A in the hook layer 804 and smaller loops 806A in the loop layer 806, such that when the hook layer 804 and the loop layer 806 are pressed together the hooks 804A catch in the loops 806A to fasten or bind the two layers (804 and 806), temporarily. The two layers (804 and 806) are separated by pulling or peeling apart the loop layer 806 from the hook layer 804. The respective backs of the hook layer 804 and the loop layer 806 comprise a first layer of adhesive 808 coupled to the hook layer 804 and a second layer of adhesive 810 coupled to the loop layer 806. The overhead adhesive patch 800 is arranged to attach to a strap arrangement (such as the strap arrangement 300 of FIG. 5A) by the second layer of adhesive 810 coupled to the loop layer 806 and to a distal surface 802A of the wearable device 802 by the first layer of adhesive 808 coupled to the hook layer 804. Notably, the wearable device 802 may be placed onto a body of a user using the overhead adhesive patch 800 that arranges the distal surface 802A of the wearable device 802 with the strap arrangement, so that a proximal surface 802B of the wearable device 802 contacts the body of the user. The use of Velcro® fasteners allows for repositioning of the wearable device as well as detachment.

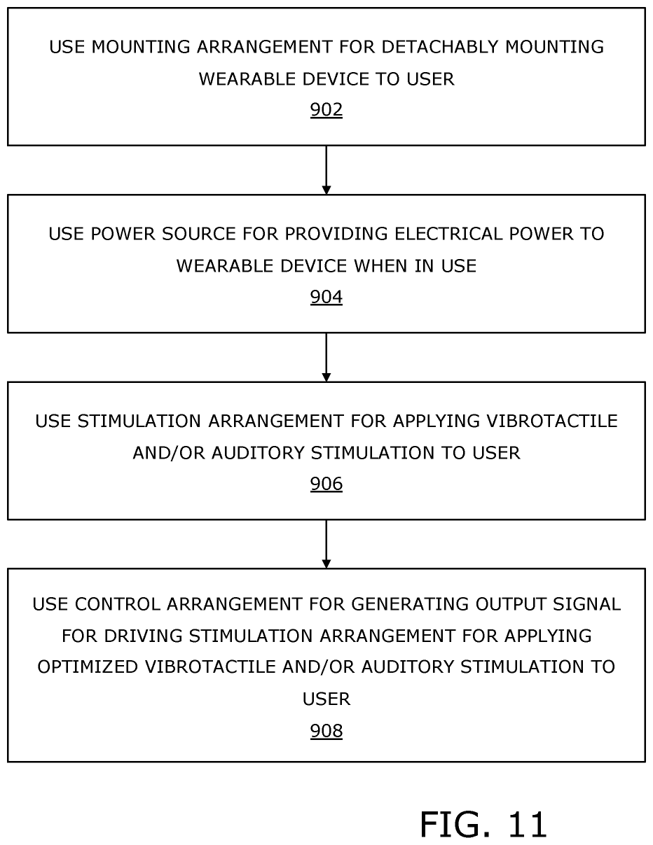

Referring to FIG. 11, there is shown a flowchart of a method for (namely, a method of) operating the wearable device 100 for mitigating symptoms of a neurological condition of a given user. At a step 902, a mounting arrangement 102 is used for detachably mounting the wearable device 100 to the user. At a step 904, a power source 104 is used for providing electrical power to the wearable device 100 when in use. At a step 906, a stimulation arrangement 106 is used for applying vibrotactile stimulation, auditory stimulation and an electrical stimulation, or any combination of these, to the given user, beneficially together with cueing. At a step 908, a control arrangement 108 is used for generating an output signal for driving the stimulation arrangement 106 for applying the stimulation with cueing to the given user. Moreover, the wearable device 100 is configured to excite the stimulation arrangement 106 during the cueing period, as illustrated in FIGS. 1 and 2, with pulsing-period stimulation and resting-period stimulation.

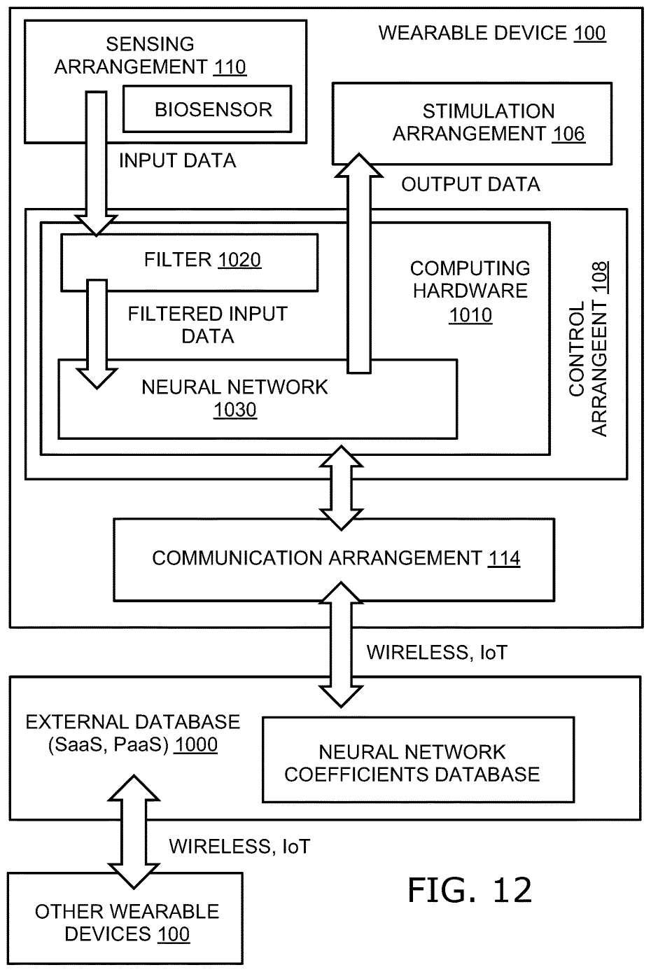

The steps 902, 904, 906 and 908 are only illustrative, and other alternatives can also be provided where one or more steps are added, one or more steps are removed, or one or more steps are provided in a different sequence without departing from the scope of the claims herein. Next, operation of the control arrangement 108 will be described in greater detail. Referring to FIG. 12, within the wearable device 100, the control arrangement 108 is configured to receive input data from the sensor arrangement 110 and also to provide output data (for example output drive signal) to the stimulation arrangement 106. Optionally, the control arrangement 108 is configured to communicate via the communication arrangement 114 to an external database 1000; the external database 1000 is beneficially implemented as a cloud-based server arrangement configured to function as a software-as-a-service (SaaS) or as a platform-as-a-service (PaaS). The communication arrangement 114 is conveniently implemented wirelessly, for example via Internet-of-Things (IoT). The control arrangement 108 includes computing hardware 1010, as aforementioned, that is configured to execute one or more software products to implement one or more data processing algorithms. The one or more software products are optionally provided already loaded into data memory of the control arrangement 108 prior to the wearable device 100 being provided to the user. Optionally, one or more software products are downloadable from the external database 1000 to the wearable device 100, for example to provide software updates, neural network configuration data and similar. In operation, the computing hardware 1010 is configured to use a filter 1020 to filter the input data received from the sensing arrangement 108 to remove interference and noise therefrom to generate corresponding filtered input data (Vᵢₙ(t)); conveniently, such filtering may be achieved by using at least one of:
(i) digital recursive filters, wherein the recursive filters are optionally adaptable to attenuate specific types of interference and noise present in the input data (Vᵢₙ(t));
(ii) Fast Fourier Transform filters for converting the input data (Vᵢₙ(t)) via a Fast Fourier Transform (FFT) algorithm to generate corresponding spectral Fourier coefficients, and then removing contributions from specific spectral Fourier coefficients corresponding to interference and noise; the remaining spectral Fourier coefficients after removal of the spectral Fourier coefficients corresponding to noise and interference are then used in further computations executed by the computing hardware 1010; and
(iii) Hidden Markov Models (HMM) filters that are configured to apply a signal processing model to separate desired components of the input data from noise and interference present in the input data (Vᵢₙ(t)); and then generating the filtered input data from the desired components.

The digital recursive filters, Fast Fourier Transform filters, and Hidden Markov Models are beneficially implemented as algorithms that are implemented in software routines that are executable on the computing hardware 1010. As aforesaid, the filtered input data is provided to other algorithms implemented in executable software running on the computing hardware 1010.

The computing hardware 1010 is also beneficially configured to implement a neural network 1030, for example implemented using neural network software algorithms. The neural network 1030 has neural network weighting coefficients that are beneficially configurable for specific conditions of the user. For example, the user (or a carer responsible for the user) uses a software application (“app”) to upload data describing the user's specific individual neurological medical condition together with other supporting data (for example, the user's medical history, medications that the user regularly consumes, trauma that the user may have experienced and so forth) to the external database 1000, wherein the external database 1000 then identifies corresponding neural network weighting coefficients that are optimal to use for assisting the user. For example, the external database 1000 stores a series of pre-prepared templates of neural network weighting coefficients, wherein a particular template that best matches the user's condition is downloaded to the user's wearable device 100. Thus, the network weighting coefficients are then downloaded to the computing hardware 1010 of the wearable device 100 via the communication arrangement 114, and then used by the neural network 1030 to analyse the filtered input data (Vᵢₙ(t) to generate the output data (Vₒᵤₜ(t) to drive the stimulation arrangement 106 of the user's wearable device 100. By such an approach, operating parameters of the wearable device 100 are optimized and customized to the user. By including the neural network 1030 locally within the wearable device 100 and configuring the neural network 1030 specifically for the needs of the user, the wearable device 100 is able to function autonomously, even when the communication arrangement 114 is not able to communicate in real-time with the external database 1000. Optionally, the neural network 1030 is coupled to the filter 1020 to be able to control at least one of: settings of its recursive filters, choices of spectral Fourier coefficients to be removed or included in the filtered input data, settings of the Hidden Markov Models. Optionally, the wearable device 100 is configured to operate in a plurality of modes, that are described in more detail elsewhere in the present disclosure. One of the modes concerns operating the wearable device 100 for a period of time without any stimulation via the stimulation arrangement 106 being used, thereby providing the neural network 1030 with an opportunity to assess sources of noise and interference afflicting the wearable device 100, and to configure the filter 1020 accordingly. Once the filter 1020 has been configured, the wearable device 100 is then able to switch to its normal configuration of operation where the wearable device 100 provides stimulation during the cueing period to the user.

It will be appreciated that the user's medical condition may vary as a function of time, as well as a function of environmental conditions and other medical conditions of the user (for example, blood sugar level in an event that the user is diabetically challenged). As a result, the configuration of the neural network 1030 may be required to change as a function of time to track adaptively to changes in the user's neurological condition.

Beneficially, the wearable device 100 is configured to be adaptive and modify its stimulation applied to the user. Such an adaptation is both advantageous for the user and for operation of the external database 1000. Optionally, the neural network 1030 is configured to apply small perturbations to the output data from one cueing period to another cueing period, to investigate how optimized the coefficients of the neural network 1030 are to address needs of the user. In an event that a particular given perturbation enables the wearable device 100 to assist the user more effectively, the neural network 1030 is able to reconfigure itself to take the perturbation into account, and also to communicate to the external database 1000 information indicative of the benefit or disadvantage of utilizing the perturbation. The external database 1000 is able, for example when results from other wearable devices 100 also confirm that the perturbation is beneficial for other users, to update the neural network coefficients that are available for download from the external database 1000 ; in other words, the external database 1000 is able to determine “best practice” when providing treatment to the user. The wearable device 100 is configured to use biological data, for example biological sensor data, for selecting a preferred set of neural network coefficients from the external database 1000 for use in the control arrangement 108 of the wearable device 100. Moreover, input data generated from one or more biosensors included in the sensor arrangement 110 is used, when the wearable device 100 is in operation assisting the user, as input data to the neural network 1030 and influences generation of the output data that is applied to the stimulation arrangement 106.

Optionally implementations of the wearable device 100 will next be described, for example in respect of a treatment dose provided to the user by way of exciting the stimulation arrangement 106.

The control arrangement 108 is beneficially configured to generate the output signal for exciting the stimulation arrangement 106 for applying a range of types of stimulation to the user, wherein the control arrangement 108 is configured to adjust a stimulation dose applied to the user based on the user's biological parameters, response, and preferences. The control arrangement 108 is configured to apply a variable stimulation dose by adjusting at least one of: a vibrotactile stimulation frequency, an amplitude of vibrotactile stimulation, a duration of vibrotactile stimulation, an auditory stimulation frequency, an amplitude of auditory stimulation, a duration of auditory stimulation, a pattern of the vibrotactile stimulation applied by the stimulation arrangement 106 to the user, and a pattern of the auditory stimulation applied by the stimulation arrangement 106 to the user. Optionally, the control arrangement 108 is configured to implement machine learning algorithms (namely, artificial intelligence (AI) algorithms) for identifying patterns or trends in the user's response to the applied stimulation dose provided by the stimulation arrangement 106 to the user, and to adjust the stimulation dose accordingly to provide improved mitigation of the symptoms of the neurological condition afflicting the user. Beneficially, the stimulation arrangement 106 includes multiple actuators, as aforementioned, which are configurable to provide different vibrotactile and/or auditory stimulation dose characteristics, allowing for a variety of stimulation patterns and associated amplitudes to be applied to the user. Optionally, the wearable device 100 includes the aforementioned user interface arrangement 112 for the user to provide real-time feedback in response to the stimulation dose being applied thereto, allowing for manual adjustments to the applied stimulation to be made by using the user interface arrangement 112.

The wearable device 100 is therefore configurable to implement a method for calibrating the stimulation, wherein the method includes steps of:
(i) performing an initial assessment of the user's symptoms and biological parameters;
(ii) applying a test stimulation dose with cueing to the user via the stimulation arrangement 106;
(iii) measuring the user's response to the test stimulation dose and cueing;
(iv) adjusting the dose based on the user's response to the test stimulation dose; and
(v) iterating around steps (iii) and (iv) to optimize the test stimulation dose with cueing to mitigate the symptoms of the user's neurological condition.

The control arrangement 108 is thereby configured to implement an adaptive therapy mode of operation, for automatically adjusting the stimulation dose in response to changes in the user's neurological condition or symptoms over time. In other words, the stimulation dose may be made adaptable as a function of time as the symptoms of the user's neurological condition change, for example as a function of muscle fatigue, changes in the user's biological parameters, other treatments being provided concurrently to the user (for example, consumption of pharmaceutical drugs and such like).

As aforementioned, the wearable device 100 beneficially includes a biosensor arrangement that includes multiple biosensors for detecting various biological parameters of the user, thereby providing a comprehensive assessment of the user's biological condition and response to varying the stimulation dose, whether vibrotactile stimulation dose or auditory stimulation dose, or both.

As aforementioned, the control arrangement 108 is beneficially configured to receive updates, allowing for the one or more algorithms of the control arrangement 108 to be adaptively updated, for example for implementing stimulation dose adjustments, and personalization based on new research findings or feedback from other users.

Optionally, the wearable device 100 is configured to apply the stimulation dose in a series of therapy sessions, wherein each session potentially has a different stimulation dose based on the user's condition, preferences, or feedback.

Optionally, the control arrangement 108 is configured to include one or more safety features to prevent overstimulation or discomfort to the user, such as limiting the maximum amplitude, duration, or frequency of the stimulation provided by the stimulation arrangement 106 to the user.

Optionally, the wearable device 100 is configured to function with a remote control or a mobile communication device software application that allows the user or a healthcare professional to easily adjust the stimulation dose provided by the wearable device 100, for example during therapy session parameters, or user preferences, but not limited hereto.

Optionally, the control arrangement 108 includes a machine learning-based predictive model that anticipates changes in the user's neurological condition, thereby pre-emptively adjusting the stimulation dose for providing optimal mitigation of the symptoms afflicting the user.

In FIG. 13, there is shown a graph having frequency plotted and an abscissa axis and acceleration [G], namely vibrotactile stimulation plotted along an ordinate axis. The graph includes a typical response of the linear resonant actuator to minimum and maximum linear resonant actuator drive provided in the output signal from the control arrangement 108: corresponding vibrotactile stimulation provided by the linear resonant actuator is denoted by “LRA Spectrum @ Min Magnitude”, “LRA Spectrum @ Max Magnitude”, respectively. It will be appreciated from FIG. 13 that the linear resonant actuator has a resonant frequency (“natural frequency”) of approximately 200 Hz. The linear resonant actuator provides appreciable efficiency of operation at frequencies below 200 Hz that enables the wearable device 100 to function over a frequency range from 80 Hz to 200 Hz, for example; most efficient operation is achieved when operating near the resonant frequency. By varying the magnitude of the output signal, the magnitude of the vibrotactile stimulation may be varied for a given frequency of vibrotactile stimulation, for example for 67 Hz and 167 Hz as shown. In comparison, operation of an eccentric rotating mass (ERM) actuator is denoted by “ERM”, wherein the vibrotactile stimulation vs. frequency is just a single curve (namely, the amplitude of vibrotactile stimulation is not independently adjustable relative to the frequency of the vibrotactile stimulation).

It will be appreciated that the aforesaid embodiments of the present disclosure are examples of implementing the present invention. It will be appreciated that other embodiments are feasible that fall within the scope of the herewith appended claims.

1. A wearable device for mitigating symptoms of a neurological condition of a user, wherein the wearable device includes:
a mounting arrangement for detachably mounting the wearable device to the user;
a power source for providing electrical power to the wearable device when in use;
a stimulation arrangement for applying a stimulation to the user,
wherein the stimulation includes vibrotactile stimulation or auditory stimulation, or both vibrotactile stimulation and auditory stimulation;
a sensor arrangement for sensing characteristics of the user to generate an input signal; wherein the sensor arrangement includes one or more motion sensors and/or one or more biosensors to sense a motion patterns and/or a biological patterns of the user, wherein the motion patterns and/or biological patterns are associated with the symptoms of the neurological condition of the user;
a control arrangement for processing the input signal to generate a corresponding output signal for driving the stimulation arrangement for applying the stimulation to the user,
wherein the wearable device is configured to excite the stimulation arrangement to provide pulsing period stimulation during a pulsing period and to provide resting period stimulation during a resting period, wherein the pulsing period stimulation is greater in amplitude than the resting period stimulation, wherein the pulsing period and the resting period are included within a cueing period, wherein the wearable device is configured to provide a concatenated series of such cueing periods, and wherein the wearable device is configured to provide cueing.

2. A wearable device of claim 1, wherein the stimulation is limited to a specific part of the user's body.

3. A wearable device of claim 1, wherein the resting period stimulation has an amplitude of substantially zero.

4. A wearable device of claim 1, wherein the one or more biosensors includes at least one of: skin-contacting electrodes for contacting onto the user's skin for sensing at least one of: nerve signals, skin characteristics, cardiac signals, skin conductivity, skin impedance, user body temperature.

5. A wearable device of claim 1, wherein the one or more biosensors and the one or more motion sensors include at least one of: a temperature sensor, a skin impedance sensor, a galvanic skin response sensor, a skin conductance sensor, an EDA (Electrodermal Activity) sensor, a heart rate sensor (ECG), a microphone, an electrostatic skin contact sensor, an accelerometer, an inclinometer, a gyroscopic sensor.

6. A wearable device of claim 1, wherein the one or more motion sensors includes at least one of: an accelerometer, an inclinometer, a gyroscopic sensor.

7. A wearable device of claim 1, wherein the control arrangement is configured to generate the output signal to excite the stimulation arrangement to generate the vibrotactile stimulation within a frequency range of 20 Hz to 500 Hz during the pulsing period, optionally within a frequency range of 30 Hz to 300 Hz, optionally with a frequency range of 80 Hz to 200 Hz, and
wherein the control arrangement is configured to implement the cueing period to have a duration in a range of 0.2 seconds to 5 seconds, optionally a duration in a range of 0.2 seconds to 4 seconds.

8. (canceled)

9. A wearable device of e claim 1, wherein the stimulation arrangement is configured to apply the vibrotactile stimulation during the pulsing period having a period to have a frequency that is independently adjustable to an amplitude of the vibrotactile stimulation.

10. A wearable device of claim 1, wherein the stimulation arrangement includes at least one actuator that is vibrationally coupled when in use to a skin region of the user.

11. A wearable device of claim 10, wherein the at least one actuator includes at least one of: a linear resonant actuator an ERM DC motor, an ERM BLDC motor, a piezo-electric actuator.

12. A wearable device of claim 1, wherein the at least one actuator includes a plurality of linear resonant actuators whose resonant frequencies are mutually different, to provide the stimulation arrangement in aggregate with an extended frequency operating range.

13. A wearable device of claim 1, further including an alarm arrangement that is configured to provide a stimulation alarm alert in an event of a condition arising needing the user's attention, wherein the stimulation alarm alert is provided by at least one of: a vibrotactile stimulation alarm alert, an auditory stimulation alarm alert.

14. A wearable device of claim 1, wherein the wearable device further includes a user interface arrangement that is configured to be used by the user for adjusting operating parameters of the wearable device, wherein the operating parameters include at least one of: a duration of the pulsing period for vibrotactile stimulation, a duration of the resting period for vibrotactile stimulation, a duration of the cueing period, an amplitude of the vibrotactile stimulation applied to the user during the pulsing period for vibrotactile stimulation, a frequency of the vibrotactile stimulation applied to the user during the pulsing period for vibrotactile stimulation, a duration of the pulsing period for auditory stimulation, a duration of the resting period of auditory stimulation, an amplitude of the auditory stimulation during the pulsing period for auditory stimulation, whether or not to provide a vibrotactile stimulation alarm alert and/or an auditory stimulation alarm alert during the pulsing period or prior thereto.

15. A wearable device of claim 1, wherein the wearable device is configured to function in a mode selected from any of:
(i) a sensing-only mode, wherein the wearable device is configured to use the sensor arrangement to sense motion patterns and/or biological patterns of the user in the input signal and, disabled from providing vibrotactile stimulation and/or auditory stimulation;
(ii) an acting-only mode, wherein the wearable device is configured to provide vibrotactile stimulation and/or auditory stimulation according to a defined pattern, optionally a user-defined pattern, and not in response to information provided by the sensor arrangement;
(iii) a sensing-acting mode, wherein the wearable device is configured to use the sensor arrangement to sense movement and/or biological parameters of the user in the input data, and to process the input data in the control arrangement to provide corresponding output data to the stimulation arrangement to apply stimulation to the user;
(iv) a manner mode, wherein the wearable device is user-adjustable to reduce an amplitude of the vibrotactile stimulation and/or the auditory stimulation for a temporary duration; and
(v) a synchronization mode, wherein a plurality of the wearable device function to synchronize their respective cueing periods.

16. A wearable device of claim 14, wherein the control arrangement includes a computing arrangement for executing at least one algorithm for processing the input signal to determine a degree of the symptoms of the neurological condition of the user, and
wherein the at least one algorithm includes at least one: a defined deterministic control algorithm, a neural network, a Hidden Markov Model, a Boltzmann machine, an adaptive neural network, and
wherein the user interface arrangement is configured to be used for adaptively adjusting the at least one algorithm being executed by the control arrangement.

17. (canceled)

18. (canceled)

19. A wearable device of claim 1, wherein the control arrangement is configured to be coupled via a communication arrangement to at least one of:
(i) another wearable device to transfer operating parameters such as user settings to the another wearable device, so that the wearable device and the another wearable device may be used alternately by the user;
(ii) a remote database or server arrangement providing a software-as-a-service (SaaS) or platform-as-a-service (PaaS) for providing for at least one of: updating software of the control arrangement for operating the wearable device, for recording a record of treatment provided by the wearable device to the user, for providing remote monitoring of operation of the wearable device; and
(iii) other such wearable devices, optionally for mutually synchronising operation of the wearable devices.

20. A wearable device of claim 1, wherein the mounting arrangement is configured for securing the wearable device to at least one region of the user, including: a neck region of the user, a chest region of the user, a sternum region of the user, a limb region (of the user, a hip region of the user, a wrist region of the user, a head region of the user.

21. A wearable device of claim 20, wherein the mounting arrangement includes a strap arrangement that is manufactured from at least one of: woven cloth, flexible tape, cotton cloth, polyester cloth, polypropylene cloth, Kevlar® cloth, Carbon fibre cloth, polyamide cloth, silk cloth, linen cloth, and
wherein the strap arrangement is adjustable to the user by using at least one of: adhesive fasteners, magnetic fasteners, Velcro®-type fasteners, pop-rivet fasteners, buckle fasteners, cloth clamps.

22. (canceled)

23. A method for operating a wearable device for mitigating symptoms of a neurological condition of a user, wherein the method includes:
using a mounting arrangement for detachably mounting the wearable device to the user;
using a power source for providing electrical power to the wearable device when in use; and
using a stimulation arrangement for applying stimulation to the user;
wherein the sensor arrangement includes one or more motion sensors and/or one or more biosensors to sense a motion patterns and/or a biological patterns of the user,
wherein the motion patterns and/or biological patterns are associated with the symptoms of the neurological condition of the user;
using a control arrangement for generating an output signal for driving the stimulation arrangement for applying the stimulation to the user,
wherein the wearable device is configured to excite the stimulation arrangement to provide pulsing period stimulation during a pulsing period and to provide resting period stimulation during a resting period, wherein the pulsing period stimulation is greater in amplitude than the resting period stimulation wherein the pulsing period and the resting period are included within a cueing period, wherein the wearable device is configured to provide a concatenated series of such cueing periods, and wherein the wearable device is configured to provide cueing.

24. A software product that is executable on computing hardware the control arrangement of the wearable device, to implement the method of claim 23.
